(19) United States
(12) Patent Application Publication
(10) Pub. No.: US 2025/0241804 A1
(43) Pub. Date: Jul. 31, 2025

(54) SUBSTRATES TREATED WITH SOLUTIONS CONTAINING CYCLODEXTRINS FOR IMPARTING ODOR CONTROL TO MATERIALS USED IN ABSORBENT PRODUCTS

(71) Applicant: Rayonier Advanced Materials Inc., Jacksonville, FL (US)

(72) Inventors: Harry CHMIELEWSKI, Jacksonville, FL (US); Tina MURGUIA, Jacksonville, FL (US); Mike PEACH, Jacksonville, FL (US); Priyanka BHATTACHARYA, Jacksonville, FL (US); Susan SHINALL, Jacksonville, FL (US); Larissa FENN, Jacksonville, FL (US)

(73) Assignee: Rayonier Advanced Materials Inc., Jacksonville, FL (US)

(21) Appl. No.: 18/858,128

(22) PCT Filed: Apr. 17, 2023
(86) PCT No.: PCT/US2023/018821

§ 371 Date: Oct. 18, 2024

Related U.S. Application Data
(60) Provisional application No. 63/332,443, filed on Apr. 19, 2022.

(51) Int. Cl. A61F 13/84

(52) U.S. Cl. CPC A61F 13/8405; A61F 2013/8435

(57) ABSTRACT

Solutions containing cyclodextrins for imparting odor control to materials used in absorbent products and associated products and methods. The solutions containing cyclodextrins for imparting odor control to materials used in absorbent products contain an optional polar organic solvent, water, cyclodextrin, and one or more odor-controlling compounds. The solutions can be used to uniformly apply a homogeneous solution of a cyclodextrin complex onto a solid surface whereby the cyclodextrin complex precipitates in situ on the solid surface. The cyclodextrin complex precipitates deposited from these solutions rapidly release odor-masking scents when hydrated with urine and can be deposited on substrates more effectively than cyclodextrin complexes in solid form.

### TECHNICAL FIELD

The present disclosure relates to solutions containing cyclodextrins for imparting odor control to materials used in absorbent products, substrates treated with solutions containing cyclodextrins for imparting odor control to materials used in absorbent products, and associated products and methods.

### BACKGROUND

Absorbent products, such as adult incontinence liners, bladder control pads, diapers, pull-up underwear, etc., should eliminate the odor of fresh urine in a short amount of time. Such products are often single-use products that are worn in public. Their users value odor protection to avoid any possibility of embarrassment. Even though many commercially available products claim to provide some type of odor control, these commercially available products can be ineffective at controlling the odor of fresh urine at the moment of use of the product. The odor of fresh urine at body temperature can often be easily detected on these absorbent products. The urine of older individuals can be particularly noticeable. Therefore, the industry still desires more effective odor control, especially in absorbent products.

Many have attempted to solve the problems associated with controlling odors in absorbent products. In some of the main approaches, odor control involves inhibiting the growth of odor-causing bacteria and adsorbing odoriferous molecules on high-surface-area adsorbents. However, such conventional technologies are generally too slow and/or ineffective at reducing the odor of fresh urine in a short amount of time.

In another approach, cyclodextrins are used for odor control in absorbent products. For instance, U.S. Pat. No. 5,429,628 discloses the use of free, uncomplexed cyclodextrins to effectively absorb malodor. U.S. Pat. No. 5,429,628 uses small particle size cyclodextrin to increase the surface area available to absorb odoriferous molecules, and thus improve odor absorption efficacy. U.S. Pat. No. 5,429,628 also teaches perfume/cyclodextrin complexes formed either by bringing the perfume and cyclodextrin together in a suitable solvent, such as water, or, preferably, by kneading and/or slurrying the ingredients together in the presence of a minimal amount of solvent, preferably water. The kneading/slurrying method is particularly desirable because the method produces smaller solid particle sizes. After freeze-drying or spray-drying, the solid complexes can be ground into particles that have a particle size of less than about 10 μm. However, care must be given when grinding to not generate a workplace safety hazard with respirable particles.

Further, the use of aqueous odor-absorbing solutions for use on odoriferous surfaces has also been described, for example, in U.S. Pat. No. 6,284,231. The composition of solutions disclosed in U.S. Pat. No. 6,284,231 comprises from about 0.1% to about 20% of a solubilized, water-soluble, uncomplexed cyclodextrin, an effective amount of at least one ingredient to improve the odor-absorbing performance of the cyclodextrin and a water-soluble antimicrobial active. These solutions are typically provided in a spray dispenser and have been commercialized under the trade name FEBREZE®.

Even though conventional technology provides cyclodextrins that can generally be used to absorb odors, it has not been straightforward to incorporate fine, dry particles of a cyclodextrin complex into an absorbent article. The dry complex powder can be sprinkled, mixed, or distributed onto the absorbent product. However, in practice, these methods of application have not been satisfactory. The immobilization of a cyclodextrin/perfume complex should not involve an excessive amount of water to avoid premature release of the perfume. Water-soluble binders, e.g., a low melting temperature polymeric binder such as polyethylene glycol, have been disclosed. Another disclosed method is the application of a cyclodextrin/perfume slurry directly to the absorbent product with a drying step. However, in practice, these slurries are difficult to handle, dispense, and uniformly apply.

To create a cyclodextrin complex in the form of a low-viscosity liquid that can be more easily handled during the manufacturing of absorbent articles, and to avoid settling of insoluble cyclodextrin particles, U.S. Pat. No. 9,592,168 teaches an absorbent article comprising an inclusion complex of cyclodextrin with an organic compound made using colloidal milling and spray drying processes, whereby the inclusion complex is dispersed in a matrix comprising polysiloxane oil. The creation of dry particles of colloidal dimensions allowed for the production of a homogeneous dispersion of solid particles that were used in the preparation of absorbent articles.

U.S. Pat. No. 5,429,628 provides examples of applying cyclodextrin to Kraft cellulose fibers by dissolving cyclodextrin in water at elevated temperatures to form a clear solution and spraying the solution onto the fibers. All of the examples require drying of the water carrier from the substrate either by air or oven drying or freeze-drying. The low solubility of the cyclodextrin requires a high solution add-on to achieve an effective level of performance, and the high water content of the solution requires drying of the cellulose fiber to prevent microbial growth during storage. Fluff pulp typically contains 6-8% water after drying. Microbial growth can occur if the water content increases into a range of about 13-15%. Therefore, all of these examples are impractical without an additional drying step.

U.S. Pat. No. 5,571,782 similarly teaches the use of an effective amount of

cyclodextrin/active complexes in the form of solid particles having particle sizes below about 12 um for fast release of the perfume active when wetted. Handling of these solid particles is straightforward in fabric softener and solid detergent compositions but can be awkward and problematic when attempting to apply the solid particles to absorbent materials and/or absorbent products.

U.S. Pat. Nos. 10,183,273 and 10,427,133 claim layers of an absorbent article treated with a substituted cyclodextrin complex (i.e., a degree of substitution=0.4 to 2.5 R per glucose unit of cyclodextrin) and an odor-controlling organic compound in powder form or dispersed in a carrier to form a dispersion for application to an absorbent article. The carrier was polysiloxane oil, mineral oil, petrolatum, polyethylene glycol, and/or glycerine. U.S. Pat. Nos. 10,183,273 and 10,427,133 also describe methods of an application using cyclodextrin and an odor-controlling organic compound in a volatile solvent system containing at least 60% water, alcohols, ketones or aldehydes, hydrocarbons, or supercritical fluids. In some forms, the solution is easily pumpable or sprayable directly onto an absorbent article. However, this approach used only substituted cyclodextrins, which are believed to stack upon crystallization into a physical form that is superior to unsubstituted cyclodextrins for the rapid release of the odor-controlling compound complexed with the cyclodextrin.

U.S. Pat. No. 8,574,683 teaches a method of making a pulp sheet of odor-inhibiting

absorbent fibers comprising a cyclodextrin odor absorbent and a biocide dissolved in a non-water liquid carrier comprised of less than 20% water and applied to a pulp sheet of cellulose fibers. Many of the perfumes and essential oils included in the present disclosure, such as citral, have been shown to have biocidal activity, including in, for example, the following disclosures: K. Winska, et. al. “Essential Oils as Antimicrobial Agents—Myth or Real Alternative?” Molecules (2019) Jun; 24(11): 2130, C. Shi. et. al. “Antimicrobial Activity and Possible Mechanism of Action of Citral against Cronobacter sakazakii” PLoS One 2016; 11(7), and M, Choudhary, et. al. “Lemongrass Essential Oil Components with Antimicrobial and Anticancer Activities” Antioxidants 2021 Dec. 22; 11(1): 20.

Even though the above-referenced applications and conventional technologies can be used to reduce odors, the need still exists for a convenient way of controlling the odor of fresh urine in absorbent products at the moment of use. That is, many of the prior approaches to odor control act too slowly for reducing the odor of fresh urine in a product at the moment of use and/or require additional processing that imparts additional costs. The inventors solved the problems with conventional approaches and provide solutions that are effective at reducing the odor of fresh urine in a very short amount of time. Users of these products value odor protection to avoid any chance of embarrassment. The present disclosure, as discussed in detail below, provides ways to achieve these and other objectives by making solutions containing relatively high concentrations of cyclodextrin complexes that can be easily spray-applied to materials used in absorbent products without requiring a drying step. The moisture increase of a treated absorbent material should be <5% by weight or even <1% by weight when a drying step is not used.

### BRIEF SUMMARY

In view of the above-mentioned exemplary problems with conventional and known cyclodextrin applications, the present application provides new and improved solutions containing cyclodextrins for imparting odor control to materials used in absorbent products.

An embodiment of the present application includes a substrate comprising an odor-controlling solution applied thereupon, wherein the odor-controlling solution comprises: water, a cyclodextrin, an odor-controlling compound, and optionally, a polar organic solvent, wherein the substrate comprises at least 0.2 kg of the odor-controlling compound per 1000 kg of the substrate, and wherein the substrate has an increase in moisture content of less than 2 wt. % as a result of the odor-controlling solution having been applied thereupon.

In an embodiment of the preceding substrate, the odor-controlling solution further comprises a stabilizing additive.

In an embodiment of any of the preceding substrates, the odor-controlling solution comprises 1.5 moles to 6 moles of the odor-controlling compound per mole of the cyclodextrin.

In an embodiment of any of the preceding substrates, the odor-controlling solution has a pH greater than 9 and less than 12.5.

In an embodiment of any of the preceding substrates, the odor-controlling solution comprises about 6 wt. % to about 19 wt. % of the cyclodextrin and the odor-controlling compound.

In an embodiment of any of the preceding substrates, the odor-controlling compound is selected from the group consisting of citral, coconut fragrance oil, d-limonene, octanol, and combinations thereof.

In an embodiment of any of the preceding substrates, the substrate has an increase in moisture content of less than 1.7 wt % as a result of the odor-controlling solution having been applied thereupon.

In an embodiment of any of the preceding substrates, the odor-controlling solution comprises 3.5 moles to 4.5 moles of the odor-controlling compound per mole of the cyclodextrin, and, before the odor-controlling solution is applied to the substrate, the odor-controlling solution is mixed at a temperature at or greater than 20° C. such that a dispersion forms that contains a complex of the cyclodextrin and the odor-controlling compound.

In an embodiment of any of the preceding substrates, the odor-controlling solution comprises about 0.1 wt. % to about 14 wt. % of the stabilizing additive.

In an embodiment of any of the preceding substrates, the stabilizing additive is selected from the group consisting of corn syrup, glucose, high fructose corn syrup, trehalose, sucrose, sorbitol, amino acids, derivatives thereof, and combinations thereof.

In an embodiment of any of the preceding substrates, the stabilizing additive is selected from the group consisting of corn syrup, glucose, high fructose corn syrup, derivatives thereof, and combinations thereof, and the odor-controlling solution comprises about 2 wt. % to about 10 wt % of the stabilizing additive.

In an embodiment of any of the preceding substrates, the stabilizing additive is trehalose, and the odor-controlling solution comprises about 0.1 wt. % to about 1.0 wt % of the stabilizing additive.

In an embodiment of any of the preceding substrates, the odor-controlling solution further comprises glucose and/or corn syrup as a stabilizing additive, wherein the odor-controlling solution comprises about 2.4 wt. % to about 9.4 wt. % of the stabilizing additive, the odor-controlling solution comprises about 3.5 moles to about 6 moles of the odor-controlling compound per mole of the cyclodextrin, the odor-controlling solution has a pH greater than 9 and less than 12.5, the odor-controlling compound is a blend of compounds selected from the group consisting of a blend of citral and coconut fragrance oil, a blend of d-limonene and coconut fragrance oil, a blend of citral and d-limonene, and a blend of octanol and d-limonene, and, before the odor-controlling solution is applied to the substrate, the odor-controlling solution is mixed at a temperature at or greater than 20° C. such that a dispersion forms that contains a complex of the cyclodextrin and the odor-controlling compound, and further the substrate has an increase in moisture content of less than 1.7 wt % as a result of the odor controlling solution having been applied thereupon.

An embodiment of the present application includes an article comprising any of the embodiments of the substrate described above.

An embodiment of the present application includes a method of preparing an odor-controlling substrate, comprising: a) providing a substrate, b) forming an odor-controlling solution comprising water, a cyclodextrin, an odor-controlling compound, a stabilizing additive, and optionally, a polar organic solvent, wherein the odor-controlling solution formed in b) is a dispersion that contains a complex of the cyclodextrin and the odor-controlling compound, c) applying the odor-controlling solution onto the substrate to obtain the odor-controlling substrate, wherein, after c), the odor-controlling substrate comprises at least 0.2 kg of the odor-controlling compound per 1000 kg of the odor-controlling substrate, and wherein the substrate has an increase in moisture content of less than 1.7 wt. % as a result of the odor-controlling solution being applied thereupon.

Another embodiment of the present application includes a substrate comprising an odor-controlling solution applied thereupon wherein the odor-controlling solution comprises: water, a cyclodextrin, an odor-controlling compound, and a polar organic solvent, wherein the substrate comprises at least 0.2 kg of the odor-controlling compound per 1000 kg of the substrate.

In an embodiment of the preceding substrate, the substrate has an increase in moisture content of less than 1 wt. % as a result of the odor-controlling solution having been applied thereupon.

In an embodiment of any of the preceding substrates, the substrate is a fluff pulp in the form of a compressed sheet or an air-laid fiber form, a synthetic or cellulosic fiber spun-bond or carded nonwoven, woven fabric, or solid material, or an absorbent article, wherein, when the substrate is the solid material, the solid material is selected from the group consisting of a polymeric foam, leather, and plastic film, and wherein, the substrate material is the absorbent article, the absorbent article is an adult incontinence control pad, feminine hygiene pad, pull-up underwear, adult brief, baby diaper, or bed pad.

In an embodiment of any of the preceding substrates, the polar organic solvent is selected from the group consisting of glycerol, propylene glycol, polypropylene glycol, polyethylene glycol, and mixtures thereof.

In an embodiment of any of the preceding substrates, the cyclodextrin is an unsubstituted or substituted α-, β- or γ-cyclodextrin.

In an embodiment of any of the preceding substrates, the odor-controlling compound is at least one of an essential oil selected from the group consisting of citral, limonene, octanol, lavandin, geranium, grapefruit, linalool, citronellol, linalyl acetate, geraniol, eugenol, floracetate, cardamom, eucalyptus, juniper berry, lavender, lemon verbena, lemongrass, patchouli, rose, spearmint, tea tree, oregano, persimmon and mixtures thereof, a botanical extract, an odor-controlling substance selected from the group consisting of benzaldehyde, cinnamic aldehyde, ligustral, florhydral, and mixtures thereof, or an antimicrobial substance selected from the group consisting eucalyptus, lavender, thyme, peppermint, cajuput, cinnamon, clove, sage, tea tree oils, and mixtures thereof.

In an embodiment of any of the preceding substrates, the odor-controlling solution comprises 1.5 moles to 1.75 moles of the odor-controlling compound per mole of the cyclodextrin.

In an embodiment of any of the preceding substrates, the odor-controlling solution has a pH greater than or equal to 12.0.

An embodiment of the present application includes an article comprising any of the embodiments of the substrate described above.

An embodiment of the present application includes a method of preparing an odor-

controlling substrate, comprising: a) providing a substrate, b) applying an odor-controlling solution onto the substrate to obtain the odor-controlling substrate, wherein the odor-controlling solution comprises: a polar organic solvent, water, a cyclodextrin, and an odor-controlling compound, and wherein, after b), the odor controlling substrate comprises at least 0.2 kg of the odor controlling compound per 1000 kg of the odor controlling substrate.

In an embodiment of the preceding method, the applying the odor-controlling solution onto the substrate in b) increases moisture content of the substrate by less than 1 wt. %.

In an embodiment of any of the preceding methods, the odor-controlling solution comprises 1.5 moles to 1.75 moles of the odor-controlling compound per mole of the cyclodextrin.

In an embodiment of any of the preceding methods, the odor-controlling solution has a pH greater than or equal to 12.0.

Other objects, features, and advantages of the present invention will be apparent to those of ordinary skill in the art in view of the following detailed description of the invention and accompanying drawings.

### BRIEF DESCRIPTION OF THE DRAWINGS

The drawings as provided herein set forth some exemplary embodiments of the solutions containing cyclodextrins for imparting odor control to materials used in absorbent products of the present application, the detailed description of which follows. The drawings are merely exemplary and are not intended to limit the invention.

FIG. 1 is a chart showing the dose and response of citral for urine odor control.

FIG. 2 is a photomicrograph at 20× with 100 μm for the smallest division, which shows particles of citral/β-cyclodextrin complexes precipitated from 86° C. glycerol/water solutions at pH 12.5.

FIG. 3 is a photomicrograph at 20× with 100 μm for the smallest division, which shows particles of citral/β-cyclodextrin complexes precipitated from 86° C. glycerol/water solutions at pH 9.6.

### DETAILED DESCRIPTION

The solutions containing cyclodextrins for imparting odor control to materials used in absorbent products, and associated products and methods of the present application are now described by reference to the embodiments. The description provided herein is not intended to limit the scope of the claims, but to exemplify the variety encompassed by the present application. The embodiments are described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As discussed above, the need exists for technologies for absorbent products, such as adult incontinence liners, bladder control pads, and pull-up underwear, to eliminate the odor of fresh urine at short times. Since such products are often single-use products that are worn in public, users value odor protection to avoid any possibility of embarrassment. Even though many commercially available products claim some type of odor control, these products can be ineffective at controlling the odor of fresh urine at the moment of use of the product. The odor of fresh urine at body temperature can be easily detected on these absorbent products.

To solve the problems with conventional technology, the present disclosure uses rapid-acting odor-masking and odor-control agents that can be incorporated into a cyclodextrin complex and deposited on fluff fibers. The active ingredients are stabilized in the complex for long shelf life and released only when the fibers contact urine. Many essential oil fragrances and organic odor-control agents can be used to make stable cyclodextrin complexes that are highly soluble in polar organic and water solvent systems. These soluble complexes can be sprayed uniformly or otherwise applied to sheets of fluff fibers. In situ precipitation of the complex on the fiber surface after cooling to room temperature provides unexpectedly efficient use of the odor-masking agents and delivers excellent odor control when the fibers become hydrated with urine. Thus, the present disclosure relates to a novel solution applied at a specific add-on amount having: (1) an optimal essential oil to cyclodextrin molar ratio and solution pH; (2) a sufficient concentration of a soluble cyclodextrin complex to impart urine odor control when applied to a material used in an absorbent product; and (3) a concentration of water low enough so as not to require an additional drying step after its application.

Cyclodextrins are cyclic oligosaccharides containing six to twelve glucose units that can form inclusion complexes with host molecules that can fit wholly or partially within the cyclodextrin cavity. Inclusion complexes comprised of highly volatile essential oils and other odor-control ingredients can be deposited on the surface of a substrate in a stable dry form. A stable, dry form is needed because volatile essential oils are a costly component of the formulation and should not evaporate from a treated substrate. The host molecules, especially the scent of essential oil, can be released by the action of water and urine. The present disclosure relates to a solution whose composition, pH, and temperature are favorable for making a soluble complex comprised of cyclodextrin and an essential oil or odor control compound, such that the solution containing the soluble complex can be sprayed or otherwise applied to a material used in an absorbent product, such that the complex precipitates in situ on the surface of the material upon cooling to room temperature, and the material treated with the complex reduces the odor of body fluids such as urine, menses, and sweat.

Cyclodextrin complexes containing cyclodextrin and odor-controlling compounds tend to have low water solubility and can be difficult to uniformly apply and be retained on the surface of a solid fiber or film. Solutions containing a polar organic solvent and water at an elevated pH and temperature provide the capability to form a cyclodextrin complex in solution and also to provide the solubility required for uniform application of a homogeneous solution of a cyclodextrin complex onto a solid surface. Upon cooling to room temperature, the cyclodextrin complex precipitates in situ on the solid fiber surface. Because the cyclodextrin complex precipitates in situ on the solid fiber surface, the control of particle sizes is also not required. Typically, complexes are formed by mixing or kneading an odor-control compound and cyclodextrin together in the presence of a small amount of water. After freeze-drying or spray-drying, the complexes are ground to a particle size of less than about 10 μm. Such a process is not needed due to the in situ precipitation utilized by the presently disclosed solutions. Additionally, colloid stabilization for making and applying complex is not required. Therefore, cyclodextrin complexes can be easily prepared and applied to absorbent materials and cellulosic materials, even without requiring a drying step.

### Solutions Containing Cyclodextrins for Imparting Odor Control

The odor-controlling solutions of the present disclosure can contain water, a cyclodextrin, an odor-controlling compound, and optionally, a polar organic solvent.

In certain embodiments, the polar organic solvent can be excluded from the odor-controlling solutions, but, in other embodiments, the polar organic solvent can be included in the odor-controlling solutions. In embodiments that exclude the polar organic solvent from the odor-controlling solutions, the odor-controlling solutions can be primarily water. The amount of water, for example, can be in the range of 82 wt. % to 93 wt. % relative to the total weight of the odor-controlling solution. In certain embodiments, the water can be in the range of 75 wt. % to 95 wt. %, 80 wt. % to 95 wt. %, 80 wt. % to 90 wt. %, 82 wt. % to 93 wt. %, or about 85 wt. %, each relative to the total weight of the odor-controlling solution. In the embodiments that include the polar organic solvent, the amount of polar organic solvent can be in the range of 4 wt. % to 10 wt. %, 5 wt. % to 8 wt. %, or about 6 wt. % to about 7 wt. % and the amount of water can be in the range of 5 wt. % to 15 wt. %, 8 wt. % to 13 wt. %, or about 11 wt. % to about 12 wt. %, each relative to the total weight of the odor-controlling solution

The polar organic solvent can be any polar organic solvent capable of providing the capability to form a cyclodextrin complex in solution and also to provide the solubility required for uniform application of a homogeneous solution of a cyclodextrin complex onto a solid surface. Examples of acceptable polar organic solvents include, but are not limited to, glycerol, propylene glycol, polypropylene glycol, polyethylene glycol, and mixtures thereof. In particular, glycerol can be used as a polar organic solvent. An increase in the amount of the polar organic solvent in the solution can increase the solubility of a cyclodextrin complex in the solution. For instance, the solubility of the complex can be increased so that an effective amount of complex can be applied to a substrate without an excessive increase in moisture. TABLE 1 below shows the increase in substrate moisture for two levels of the add-on (i.e., 0.4 kg of essential oil/ADMT fluff pulp and 2.0 kg of essential oil/ADMT fluff pulp) as a function of glycerol concentration.

TABLE 1

| | | | | | | Solution | | | | | |
|---|---|---|---|---|---|---|---|---|---|---|---|
| | Target Citral | | Total Solids | Liquid | Liquid | Solubility | Solution | Solution | Fluff | Glycerol | CD |
| | Add-On | Citral Loading | Add-On | Composition | Composition | (g total | Solubility | Add-on | Moisture | Add-On | Add-On |
| | (kg Citral/ | (mol Citral/mol | (g solids/ | (fraction | (fraction | solids/g | (g CD/g | (g soln/g | Increase | (kg glycerol/ | (kg CD/ |
| | ADMT fluff) | CD) | g fluff) | glycerol) | water) | solution) | solution) | fluff) | (%) | ADMT) | ADMT) |
| 1 | 0.4 | 1.50 | 0.0024 | 0.85 | 0.15 | 0.197 | 0.164 | 0.012 | 0.1% | 8.3 | 2.0 |
| 2 | 0.4 | 1.50 | 0.0024 | 0.50 | 0.50 | 0.197 | 0.164 | 0.012 | 0.5% | 4.9 | 2.0 |
| 3 | 0.4 | 1.50 | 0.0024 | 0.00 | 1.00 | 0.197 | 0.164 | 0.012 | 1.0% | 0.0 | 2.0 |
| 4 | 2.0 | 1.50 | 0.0119 | 0.85 | 0.15 | 0.197 | 0.164 | 0.061 | 0.7% | 41.3 | 9.9 |
| 5 | 2.0 | 1.50 | 0.0119 | 0.50 | 0.50 | 0.197 | 0.164 | 0.061 | 2.4% | 24.3 | 9.9 |
| 6 | 2.0 | 1.50 | 0.0119 | 0.00 | 1.00 | 0.197 | 0.164 | 0.061 | 4.9% | 0.0 | 9.9 |

At the higher add-on of 2.0 kg citral/ADMT fluff pulp shown in TABLE 1 as 5 and 6, it was not possible to keep the moisture increase under 1% for solution fractions of glycerol much under 85%. In other embodiments, a 100% water carrier has been used, such as in 3 and 6 shown in TABLE 1. It is possible to make a 100% water solution with relatively high concentrations of β-cyclodextrin at pH 12.5. Upon the addition of 1.5 moles of citral per mole of β-cyclodextrin, a fine, sprayable dispersion can be formed, in this case when the concentration of complex is at or below about 19.7%. At higher concentrations of complex, a coarse dispersion forms. This coarse dispersion settles in minutes and is very difficult to spray onto a substrate.

The cyclodextrin can be any cyclodextrin capable of forming cyclodextrin complexes with an odor-controlling compound. In certain embodiments, the cyclodextrin is a cyclodextrin containing six to twelve glucose units. For instance, cyclodextrins containing six to twelve glucose units can form inclusion complexes with many essential oils and odor control ingredients, wherein the essential oils and odor control ingredients can be released by the action of water and urine. Examples of acceptable cyclodextrins include, but are not limited to, commercially available, unsubstituted cyclodextrins (i.e. α-, β-, and γ-cyclodextrin). α-cyclodextrin contains 6 glucose units. β-cyclodextrin contains 7 glucose units. γ-cyclodextrin contains 8 glucose units. In particular, β-cyclodextrin can be used because β-cyclodextrin has a cost-performance ratio acceptable for use in most absorbent products.

As discussed above, the odor-controlling solution can form a cyclodextrin complex in a solution with an odor-controlling compound. Unsubstituted cyclodextrins can be used to prepare such complexes. In general, three types of unsubstituted cyclodextrins are commercially available (i.e. α-, β-, and γ-cyclodextrin). Of these commercially available unsubstituted cyclodextrins, β-cyclodextrin has a cost-performance ratio acceptable for use in most absorbent products. The solubility of β-cyclodextrin in water at about 20° C. is only 1.8 g/100 ml water. However, the solubility of β-cyclodextrin can be increased to 75 g/ml at 20° C. at a pH of 12.5 (pKₐ=12.2). The solubility of β-cyclodextrin in glycerol is 4.3 g/100 ml at 20° C. As shown in TABLE 2, the solubility of β-cyclodextrin and complexes of β-cyclodextrin/citral at elevated pH and temperature in an 85%/15% glycerol/water solution is of the order of 21.6 g/100 g solution or more. Solutions can also be made with propylene glycol. The solubility of β-cyclodextrin is greater in propylene glycol than in glycerol. Therefore, the solubility of cyclodextrin and complexes thereof can be increased by using a polar organic solvent/water solution, increasing the temperature of the solution, and increasing the pH.

TABLE 2
Solubility (g/100 g water)

β

| Temp | α | pH 6.5 | pH 12.5 | Υ |
|---|---|---|---|---|
| 25 | 12.8 | 1.8 | 75 | 25.6 |
| 60 | 66.2 | 9.1 | — | 129.2 |

Furthermore, even though substituted cyclodextrins have greater water solubility than unsubstituted cyclodextrins, the performance of unsubstituted cyclodextrins remains very good. The unsubstituted cyclodextrins can also be formulated into solutions at relatively high concentrations. Therefore, unsubstituted cyclodextrins can be used in the present solution.

The odor-controlling compounds can be any odor-controlling compound capable of providing odor control and/or odor masking. Examples of acceptable odor-controlling compounds include, but are not limited to, essential oils, such as citral, limonene, d-limonene, lavandin, geranium, persimmon, lemon, grapefruit, linalool, citronellol, linalyl acetate, geraniol, eugenol, floracetate, cardamom, eucalyptus, juniper berry, lavender, lemon verbena, lemongrass, patchouli, rose, spearmint, tea tree, oregano and mixtures thereof, botanical extracts such as Actiphyte™ by Lubrizol, odor-controlling substances, such as benzaldehyde, cinnamic aldehyde, ligustral, florhydral and mixtures thereof, antimicrobial substances, such as eucalyptus, lavender, thyme, peppermint, cajuput, cinnamin, clove, sage, tea tree oils, and fragrances such as coconut fragrance oil, and mixtures thereof. In particular, citral (i.e., C₁₀H₁₆O) can be used as an odor-controlling compound. Citral is an essential oil having a strong lemongrass odor that is effective to control and/or mask the odor of urine. Other scented essential oils, particularly limonene and persimmon, reactive aldehydes, particularly benzaldehyde, and thio-reducing compounds, particularly florhydral and ligustral, and fatty alcohols such as octanol, can be effective alone or in combination. In certain embodiments, the odor-controlling compound is a blend of compounds selected from the group consisting of a blend of citral and coconut fragrance oil, a blend of d-limonene and coconut fragrance oil, a blend of citral and d-limonene, and a blend of octanol and d-limonene.

The combined amount of the cyclodextrin and the odor-controlling compounds in the solution is limited by the solubility of the formed complex and is typically in the range of about 22 wt. % or less for glycerol/water solutions in a ratio of about 85/15. In certain embodiments, the amount of the β-cyclodextrin/citral complex in the solution can be 15 wt. % to 22 wt. %, 20 wt. % to 22 wt. %, and/or about 21 wt. %. In other embodiments, i.e., for a 100% water solution, when a fine precipitate forms upon the addition of citral to a β-cyclodextrin/water solution, the concentration for the complex can be lower, such as in the range of about 14 wt. % or less or even 13.8 wt. % or less. In certain embodiments, the concentration of cyclodextrin and odor-control compounds in the odor-controlling solution is in the range of about 6.4 wt. % to 13.8 wt. %. The odor-controlling solution can have a concentration of cyclodextrin and odor-control compounds in the range having an upper limit of 20 wt. %, 18 wt. %, 16 wt. %, 14 wt. %, 12 wt. %, 10 wt. %, 8 wt. %, or 6 wt. % and a lower limit of 1 wt. %, 2 wt. %, 3 wt. %, 4 wt. %, 5 wt. %, 6 wt. %, 7 wt. %, or 8 wt. %. In certain embodiments, the odor-controlling solution comprises about 6 wt. % to about 19 wt. % of the cyclodextrin, and the odor-controlling compound or the odor-controlling solution comprises about 6.4 wt. % to about 18.2 wt. % of the cyclodextrin and the odor-controlling compound. In other embodiments, the odor-controlling solution comprises about 8 wt. % to about 9 wt. % of the cyclodextrin and the odor-controlling compound. At higher concentrations such as concentrations exceeding 20 wt. %, an undesirably coarse precipitate can form.

The solution concentration of the complex is important in reducing solution add-on so as to keep the moisture increase of the substrate below about 2 wt. %, below about 1.7 wt. %, below about 1 3 wt. %, and/or below about 1 wt. %. However, the ratio of odor-controlling compound to cyclodextrin is also important for imparting good odor control performance to a substrate. For citral, the optimum molar ratio of citral to β-cyclodextrin is about 1.5-1.75 moles of citral to 1.0 mole of β-cyclodextrin. At molar ratios of about 1:1 and less, odor control performance is much reduced (at equal add-ons of citral). At molar ratios of 1.75:1 and greater, volatile, uncomplexed citral imparts unnecessary color and scent to the substrate for solutions containing about 85% glycerol. For solutions made without polar organic solvents, EO:CD mole ratios in the range of 4 to 6 allow for mixing at 50° C., and mole ratios in the range of 4 to 6 allowed mixing at 20° C. with the addition of corn syrup. In general, the molar ratio of odor-controlling compounds to cyclodextrin can be, for example, 2.5:1, 2.25:1, 2.0:1, 1.75:1, 1.5:1, 1.4, 1.3, 1.2, 1.1, and 1.0. In certain embodiments, the molar ratio of odor-controlling compounds to cyclodextrin can be 6.0:1, 5.5:1, 5.0:1, 4.5:1, 4.0:1, 3.5:1, and 3.0:1. Also, in certain embodiments, the odor-controlling solution comprises 1.5 moles to 6 moles of the odor-controlling compound per mole of the cyclodextrin. In other embodiments, the odor-controlling solution comprises 3.5 moles to 4.5 moles of the odor-controlling compound per mole of the cyclodextrin. Increasing the molar ratio of odor-controlling compounds to cyclodextrin can improve commercial processing by allowing for reduced solution mixing pH and reduced mixing temperature, especially for, but not limited to, solutions that are mostly water-based.

A higher mole ratio of citral to β-cyclodextrin allows for less use of glycerol and β-cyclodextrin to achieve a target add-on of citral (kg citral per air-dried metric ton of fluff). It also reduces the moisture increase of the fluff pulp, as shown in TABLE 3 below. The molecular weights of β-cyclodextrin and citral are 1135 g/mole (dry) and 152 g/mole, respectively. The use of Actiphytes™, Lubrizol Inc., and other more concentrated botanical essences could enable the loading of β-cyclodextrin with even higher levels of odor-control compounds.

TABLE 3

| Target | | | | |
|---|---|---|---|---|
| Citral | | | Glycerol | β-cyclodextrin |
| Add-On (kg | Mole Ratio | | Add-On | Add-On |
| citral/ADMT | (citral/β- | Moisture | (kg/ADMT | (kg/ADMT |
| fluff) | cyclodextrin) | Increase | fluff) | fluff) |
| 0.4 | 1 | 0.30% | 15.1 | 3.1 |
| 0.4 | 1.5 | 0.10% | 7.4 | 2 |

In addition, commercially available complexes, such as a citral/β-cyclodextrin complex (Wacker Chemical, CAVAMAX® W7 CITRAL Complex), can be used to impart good odor control performance to fluff pulp for solutions containing a polar organic solvent. The commercially available complexes are believed to have a minimum 8.5 wt. % citral content, which suggests a minimum citral: β-cyclodextrin mole ratio of 0.7:1. The target value of the citral: β-cyclodextrin mole ratio is unknown but it is likely to be in the range of 1.0:1. The odor-controlling solution can have a pH of greater than or equal to 12.5, 12, 11, 10, or 9.6. Below a pH of about 12, odor-control performance is greatly reduced. In certain embodiments, the pH is about 12.5. Further reducing the pH to 11 or even lower to a pH of 10 improves the ease of commercial processing. In certain embodiments, the odor-controlling solution has a pH greater than pH 9.6 and less than 12.5, a pH greater than 9 and less than 12.5, or greater than pH 10 and less than 11. Such commercially available complexes, however, have an extremely high cost, which renders their implementation cost prohibitive and impractical on a commercial scale.

The odor-controlling solutions can also optionally contain a stabilizing additive. The stabilizing additive can help disrupt the precipitation and crystallization of the dispersed complex of cyclodextrin and an odor-controlling compound. In particular, in certain embodiments, the mole ratio of the water-based solutions was increased to reduce solution concentration and prepare samples at 50° C. and pH 12.5. The stabilizing additive allows such high-mole-ratio solutions to be made at 20° C. and pH 7, such as the embodiments shown in Example 14. The reduced mixing temperature and pH allow for simplified commercial processing with excellent odor-control performance. Examples of acceptable stabilizing additives include, but are not limited to, corn syrup, glucose, high fructose corn syrup, trehalose, stabilizing osmolytes including sucrose, sorbitol, amino acids and their derivatives, and mixtures thereof. The odor-controlling solution can contain about 0.1 wt. % to about 14 wt. % of the stabilizing additives, about 4 wt. % to about 14 wt. % of the stabilizing additives, about 4 wt. % to about 10 wt. % of the stabilizing additives, about 0.1 wt. % to about 1.0 wt. %, or even 2.4 wt. % to 9.4 wt. %. In certain embodiments, the additive providing a stabilizing effect to the dispersion is corn syrup, glucose, or high fructose corn syrup at a solution concentration of about 2 wt. % to 10 wt. % or even 6.2 wt. % to 9.4 wt. %. In other embodiments, the additive providing a stabilizing effect to the dispersion is trehalose at a solution concentration of about 0.1 wt. % to 1.0 wt. %. In yet further embodiments, the additive providing a stabilizing effect to the dispersion is selected from the group consisting of protective or stabilizing osmolytes including sucrose, sorbitol, amino acids, and their derivatives.

The odor-controlling solutions described herein allow for a cyclodextrin complex to be precipitately deposited from the odor-controlling solutions and rapidly release odor-masking scents when hydrated with urine. Typical odor-controlling compounds are scented essential oils that are released from the cyclodextrin complex upon hydration of the treated fiber or film surface. The odor-controlling solution of the present disclosure can impart odor control to materials used in absorbent products. The odor-controlling solution uses rapid-acting odor-masking and odor-control agents, e.g., odor-controlling compounds, that can be incorporated into a cyclodextrin complex and deposited on fluff fibers. These fluff fibers, also known as cellulose fluff pulp, can be used as an absorbent filler material for absorbent, such as diapers, bed pads, etc. The active ingredients (i.e., the odor-controlling compounds) can be stabilized in the cyclodextrin complex for long shelf life and released only when the fibers contact urine. Essential oil fragrances and organic odor-control agents can be used to make stable cyclodextrin complexes that are highly soluble in polar organic and water solvent systems. These soluble complexes can be uniformly sprayed or otherwise applied to sheets of fluff fibers. In situ precipitation of the cyclodextrin complexes on the fiber surface after cooling to room temperature provides unexpectedly efficient use of the odor-masking agents and delivers excellent odor control when the fibers become hydrated with urine. The odor-controlling solution of the present disclosure has: (1) a sufficient concentration of a cyclodextrin complex to impart urine odor control when applied to a material used in an absorbent product; and (2) an added amount of water that is low enough so as not to require an additional drying step after its application.

The odor-controlling solution of the present disclosure for imparting odor control to materials used in absorbent products can contain an, optional, polar organic solvent, water, cyclodextrin, and one or more odor-controlling compounds. Cyclodextrin complexes containing a cyclodextrin and odor-controlling compounds have low water solubility and are difficult to uniformly apply and be retained on the surface of a solid fiber or film. Solutions comprised of a polar organic solvent and water at elevated pH and temperature provide the capability of forming a cyclodextrin complex in the solution. Such odor-controlling solutions also provide the solubility and solution stability desired for the uniform application of a homogeneous solution of a cyclodextrin complex onto a solid surface.

### Substrate Having an Odor Controlling Solution Applied Thereupon

The substrate of the present disclosure comprises an odor-controlling solution applied thereupon. The odor-controlling solution is described in detail above, the discussions of which are incorporated herein by reference.

The substrate comprises at least 0.2 kg of the odor-controlling compound per 1000 kg of the substrate. In certain embodiments, the substrate comprises at least 0.3 kg of the odor-controlling compound per 1000 kg of the substrate. In other embodiments, the substrate comprises at least 0.6 kg, 0.8 kg, 1 kg, 1.5 kg, or 2 kg of the odor-controlling compound per 1000 kg of the substrate.

Also, the substrate can have an increase in moisture content of less than 2 wt. % as a result of the odor-controlling solution having been applied thereupon. In certain embodiments, the increase in moisture content can be less than 2 wt. % such as 1.6 wt. %, 1.3 wt. %, 1 wt. %, etc., as discussed in the present disclosure. That is, even after applying a sufficient amount of the odor-controlling solution to achieve at least 0.2 kg of the odor-controlling compound per 1000 kg of the substrate, the substrate still can have an increase in moisture content of less than 2 wt. %, as measured by comparing the weight of the substrate before application of the odor controlling solution and after the application of the odor controlling solution.

The odor-controlling solution discussed above provides cyclodextrin complexes containing cyclodextrin and odor-controlling compounds. By utilizing a polar organic solvent and water at elevated pH and temperature, the cyclodextrin complex can be provided in a homogeneous solution and applied to the substrate. The cyclodextrin complexes precipitate in situ on the substrate after cooling to room temperature, but the substrate does not have a substantial increase in moisture content. Also, in certain embodiments, the cyclodextrin complexes can be dispersed in the odor-controlling solution without polar organic solvent for application to the substrate. In certain embodiments, the substrate can have an increase in moisture content of less than 0.8 wt. % as a result of the odor-controlling solution having been applied thereupon. In certain embodiments, the substrate can have an increase in moisture content of less than 5 wt. %, 4 wt. %, 3 wt. %, 2 wt. %, 1.7 wt. %, 1.6 wt. %, 0.6 wt. %, 0.4 wt. %, 0.2 wt. %, 0.13 wt. %, or 0.1 wt. %. In certain embodiments, the odor-controlling solution can contain 67 wt. % of glycerol as the polar organic solvent, 12 wt. % of the water, and 21 wt. % of the cyclodextrin and the odor-controlling compound, wherein the cyclodextrin is unsubstituted β-cyclodextrin, and wherein the odor-controlling compound is citral, wherein the citral and the unsubstituted β-cyclodextrin are present in a mole ratio of 1.5 moles of the citral per mole of the unsubstituted β-cyclodextrin, wherein the substrate is fluff pulp, wherein the substrate comprises 0.4 kg of the citral per 1000 kg of the fluff pulp, and wherein the substrate has an increase in moisture content of less than 0.13 wt. % as a result of the odor controlling solution having been applied thereupon. In certain embodiments, the odor-controlling solution can be made without polar organic solvent, 85 wt. % of the water, and 8 wt. % of the cyclodextrin and the odor-controlling compound, wherein the cyclodextrin is unsubstituted β-cyclodextrin, and wherein the odor-controlling compound is an equimolar blend of d-limonene and octanol, wherein the odor-controlling compounds and the unsubstituted β-cyclodextrin are present in a mole ratio of 4.0 total moles of the odor-controlling compounds per mole of the unsubstituted β-cyclodextrin, wherein the substrate is fluff pulp, wherein the substrate comprises 0.51 kg of the odor-controlling compounds per 1000 kg of the fluff pulp, and wherein the substrate has an increase in moisture content of less than 1.7 wt. % as a result of the odor controlling solution having been applied thereupon. There is a significant practical and economic value to limiting the moisture content of the substrate to less than about 2 wt. %. The addition of a drying step to a typical fluff pulp machine would require capital in the range of $30-40 million. Therefore, it is desirable to reduce the moisture content to the extent possible.

The substrate is not limited to any particular substrate material. For instance, the substrate can be a fluff pulp in the form of a compressed sheet or an air-laid fiber form, a synthetic or cellulosic fiber spun-bond or carded nonwoven, woven fabric, solid material, or an absorbent article. When the substrate is the solid material, the solid material is selected from the group consisting of polymeric foam, leather, and plastic film. When the substrate is an absorbent article, the absorbent article is an adult incontinence control pad, feminine hygiene pad, pull-up underwear, adult brief, baby diaper, or bed pad.

### Article Comprising the Substrate Having an Odor Controlling Solution Applied Thereupon

The articles of the present disclosure comprise the substrate discussed above, the discussion of which is incorporated herein by reference. The article is not limited, but can include, for example, articles that would benefit from the improved odor control achieved by the present disclosure. For instance, such articles can include absorbent articles such as an adult incontinence control pad, feminine hygiene pad, pull-up underwear, adult brief, baby diaper, or bed pad. Other substrates include, but are not limited to, fluff pulp in compressed sheet or air-laid fiber form, synthetic or cellulosic fiber spun-bond or carded nonwoven, woven fabric, or solid material such as polymeric foam, leather, or plastic film.

### Method of Preparing an Odor Controlling Substrate

The odor-controlling substrate can be prepared by providing a substrate, and applying an odor-controlling solution onto the substrate to obtain the odor-controlling substrate. The substrate and the odor-controlling solution are described in detail above, the discussions of which are incorporated herein by reference.

The method can further include forming an odor-controlling solution comprising water, a cyclodextrin, an odor-controlling compound, a stabilizing additive, and optionally, a polar organic solvent. The odor-controlling solution prepared in the method can form a dispersion that contains a complex of cyclodextrin and the odor-controlling compound. Because the dispersion is transiently stable, the odor-controlling solution containing the dispersion can be applied to the substrate before destabilization of the dispersion occurs. The formation of a dispersion can occur at different temperatures and pHs when different components are used in the odor-controlling solution. For example, when the stabilizing additive is corn syrup, the odor-controlling solution can be prepared at room temperature (i.e., about 20° C.) and a pH of greater than 9. When the odor-controlling solution is prepared without corn syrup, the odor-controlling solution can be prepared at a temperature greater than 50° C. and a pH of greater than 10.9. In certain embodiments, the odor-controlling solution is prepared by mixing water and a stabilizing additive (e.g., corn syrup) and then adjusting the pH of the mixture. The mixture can then be heated to the desired temperature, such as a temperature greater than 50° C., or maintained at room temperature (i.e., about 20° C.). The cyclodextrin and the odor-controlling compound can then be added to the mixture. The mixture can then be applied to a substrate.

As discussed above, the mixing of certain embodiments of the odor-controlling solution can occur at a temperature at or greater than about 50° C. to produce a dispersion of the cyclodextrin and the odor-controlling compound. However, in certain embodiments, the mixing can occur at room temperature (i.e., about 20° C.). Additionally, the odor-controlling solution can be at a reduced pH of, for example, 11.5, 11.0, 10.5, 10, 9.5, or 9. For example, the pH can be in the range of 9 to 12.5. Both the reduced temperature and pH of the odor-controlling solution individually and collectively ease commercial processing, even with a higher molar ratio of odor-controlling compounds to cyclodextrin (e.g., a citral to cyclodextrin mole ratio of 4.0 or even higher). The dispersibility at higher molar ratio of odor-controlling compounds to cyclodextrin was unexpected and allowed for increased odor-control performance. The addition of the stabilizing additive (e.g., corn syrup) also disrupts the precipitation and crystallization of the dispersed cyclodextrin complex, and reduces destabilization of the dispersion. The stabilizing additive (e.g., the smaller glucose molecules) is believed to interact with the outer surface of the chemically-similar, β-cyclodextrin complex to reduce the rate and amount of crystallization and/or precipitation of the complex. The stabilizing additive, therefore, even further improves the performance of the odor-controlling solution, while avoiding an undesirable increase in moisture content of the fluff pulp (i.e., maintaining a moisture increase of less than 2 wt %).

In certain embodiments, the odor-controlling solution can be applied onto the substrate at elevated temperature and pH. By “elevated temperature,” the disclosure means that the temperature is greater than room temperature. For example, the odor-controlling solution can be applied onto the substrate at an elevated temperature of 70° C. to 90° C. In certain embodiments, the odor-controlling solution can be applied onto the substrate at an elevated temperature 75° C. to 90° C., 80° C. to 90° C., or 83° C. to 86° C.

In an exemplary embodiment, a fluff pulp in sheet form was an untreated southern pine Kraft pulp sold under the trade name RAYFLOC® JLDE that was obtained from Rayonier Advanced Materials. A warm solution containing citral was sprayed onto a sheet of fluff pulp at a solution add-on of about 0.055 g of odor-controlling solution per g of fluff. The sheet was not dried after the application of the odor-controlling solution. The citral add-on was calculated to be 2.0 kg citral per air-dried metric ton of fluff. The moisture content of the fluff pulp increased by 0.65%, i.e., increased from 8% moisture to 8.65% moisture. It can be beneficial to keep the increase in moisture content to a level of less than 5% or even less than 1%.

Complexation of citral with β-cyclodextrin reduces evaporation of the citral when dry, but allows the release of a citral scent when the complex is hydrated with water. The exemplary embodiment had no detectable scent when dry but had an immediate and pronounced release of lemon scent when hydrated with only 2.5 g of water per g of fluff. Furthermore, this exemplary embodiment effectively masked the scent of warm urine (at 35°-40° C.) using 2.5 g of urine per g of fluff to 10 g of urine per g of fluff.

### Other Advantages Imparted to Fluff Pulp by the Solution

Glycerol used in the formulation of the solution can impart additional benefits to fluff pulp such as reduced sheet Mullen and ease of compressibility of fiberized fluff pulp.

For instance, a sheet of RAYFLOC® pulp was sprayed on one side with 0.08 g solution/g pulp of a solution of 85 wt. % glycerol and 15 wt. % water at 80° C. reduced Mullen from an average value of 1472 kPa to 1107 kPa. Bursting strength (or Mullen) is the hydrostatic pressure, applied at a controlled rate, required to produce a rupture of a conditioned test specimen. To obtain the measurement of bursting strength (or Mullen), pressure is applied to a circular area of the test specimen, 30.48 mm (1.20 inches) in diameter, at a controlled rate through a rubber diaphragm. The material being tested is initially flat and held rigidly at the circumference of the test location. The specimen is free to bulge and eventually burst as the pressure is increased. Bursting strength (or Mullen) is recorded as the pressure in kPa at rupture.

A similar treatment was found to improve the mechanical properties of an air-laid sheet of fluff pulp. The mechanical properties related to softness and handle are KES shear modulus and compression energy. The shear modulus of fluff pulp at a basis weight of 400 g/sm and a density of 0.37 g/cc was reduced by 20%, and the compression energy was reduced by 29%. These values indicate that an air-laid core of OCF (Odor Control Fiber) might be perceived as being softer than untreated fluff pulp. The Kawabata Evaluation System (KES) is used to make objective measurements of hand properties. With low forces applied, the KES instrument measures mechanical properties that correspond to the fundamental deformation of fabrics in hand manipulation. Samples are conditioned and tests were performed at laboratory atmosphere conditions of 21°±2° C. and 65%±5% RH. Samples had a width of 20 cm. Three replications are obtained for shear stiffness. In shear testing, the KES-FB1 Tensile-Shear Tester applies opposing and parallel forces to the specimen, until a maximum offset angle of 0.5° is reached. A pretension load of 50 gf/cm was applied. A lower value of shear stiffness (gf/cm*degree) indicates less resistance to shearing movement corresponding to a softer material.

### EXAMPLES

The features and advantages of the solutions containing cyclodextrins for imparting odor control to materials used in absorbent products and associated products and methods are more fully shown with respect to the following illustrative examples and embodiments.

### Example 1: Preparation of Cyclodextrin Complex in Solution

In the following example, β-cyclodextrin was obtained from Acros Organics (CAS: 7585-39-9) and Wacker Chemical under the trade name W7. Citral is a volatile lemongrass-scented essential oil that was obtained from Perfumer's Apprentice (CAS: 5392-40-5).

A solution was prepared by first blending 85 wt. % glycerol and 15 wt. % water and increasing its pH to 12.5 with sodium hydroxide. 50 g. of this mixture was heated after the pH adjustment. At about 75° C., 11.5 g. of β-cyclodextrin was added. Heating and stirring continued until the β-cyclodextrin was completely dissolved at around 86° C. At that point, 2.3 g. of citral was added (using a mole ratio of 1.5 moles citral/mole β-cyclodextrin) with continued stirring for a few minutes until a clear, water-white solution was obtained. This solution contained 21.6 g of β-cyclodextrin and citral per 100 g of solution, 66.7 g of glycerol per 100 g solution, and 11.8 g of water per 100 g. solution. The water content of the β-cyclodextrin (up to 10% by weight) was not considered in these calculations.

### Example 2: pH Determination

In the following example, the pH of untreated RAYFLOC® fluff from Rayonier Performance Fibers, LLC was compared to an exemplary embodiment, 31-5 OCF (Odor Control Fiber), that was treated with a solution of the present disclosure. The solution applied to the 31-5 OCF sample contained 66.6 wt. % glycerol, 11.8 wt. % water, 18.0 wt. % β-cyclodextrin, and 3.6 wt. % citral (i.e., 1.5:1 citral:cyclodextrin molar ratio). The pH of the glycerol/water solution was adjusted to a pH of 12.5 at room temperature using sodium hydroxide. The 31-5 OCF sample was made using a solution add-on of 0.011 grams solution per gram of fluff at 86° C. to provide a citral add-on of 0.4 kg citral per air-dried metric ton of fluff. Using the test method below, the 31-5 OCF sample had a pH of 5.8 and the untreated RAYFLOC® fluff had a pH of 6.0, as shown in TABLE 4 below. The use of a relatively high pH solution had a negligible effect on the pH of the 31-5 OCF.

TABLE 4

| | pH |
|---|---|
| Deionized water | 6.5-7.0 |
| RAYFLOC ® fluff pulp | 6 |
| 31-5 OCF | 5.8 |

The method for determining the pH of the fibers is provided as follows. The pH of the fiber was determined by placing two grams of a defiberized sample into a 500 mL Erlenmeyer flask. 100 grams of distilled water at pH 6.5-7.0 was added. The flask was glass-stoppered and shaken until a thick slurry was obtained. An additional 100 grams of distilled water was added to the flask before it was stopped and briefly shaken at about 15-minute intervals until the sample was completely slurried. After about one hour, about 50 mL of the slurry was poured into a clean 150 ml beaker. This portion was used to rinse the beaker and was discarded. The remainder of the slurry was then poured into a beaker and pH was determined with a previously calibrated pH meter.

### Example 3: Dose/Response of Citral for Urine Odor Control

In the following example, the dose and response of citral for urine odor control in exemplary embodiments were determined.

Using the solution described in Example 2, exemplary embodiments were prepared whereby the amounts of citral add-on were varied over a range of 0.1 to 3.3 kg citral per air-dried metric ton of fluff. The citral add-on was varied by adjusting the solution add-on. Fluff pulp in sheet form was an untreated southern pine Kraft pulp sold under the trade name RAYFLOC® JLDE that was obtained from Rayonier Advanced Materials of Jesup, GA. The following TABLE 5 summarizes the variations used to determine the dose and response of citral for urine odor control.

TABLE 5

| | | | | | | TOTAL | Water (control) | Urine (control) |
|---|---|---|---|---|---|---|---|---|
| | Citral:CD | Citral add-on | | Mix Temp | Appl'n | ODOR | vs. Urine | vs. Urine |
| | mole ratio | (kg/ADMT) | pH | (C.) | Temp (C.) | SCORE | ODOR SCORE | ODOR SCORE |
| 25-1 | 1.5 | 3.3 | 12.5 | 86° | 86° | +48 | +12 | +36 |
| 31-1 | 1.5 | 2.0 | 12.5 | 86° | 86° | +56 | +24 | +32 |
| 31-4 | 1.5 | 0.8 | 12.5 | 86° | 86° | +52 | +20 | +32 |
| 31-5 | 1.5 | 0.4 | 12.5 | 86° | 86° | +52 | +24 | +28 |
| 37-1 | 1.5 | 0.2 | 12.5 | 86° | 86° | +38 | +20 | +18 |
| 37-2 | 1.5 | 0.1 | 12.5 | 86° | 86° | −18 | −36 | +18 |
| theoretical | | 0 | | | | −36 | −36 | 0 |

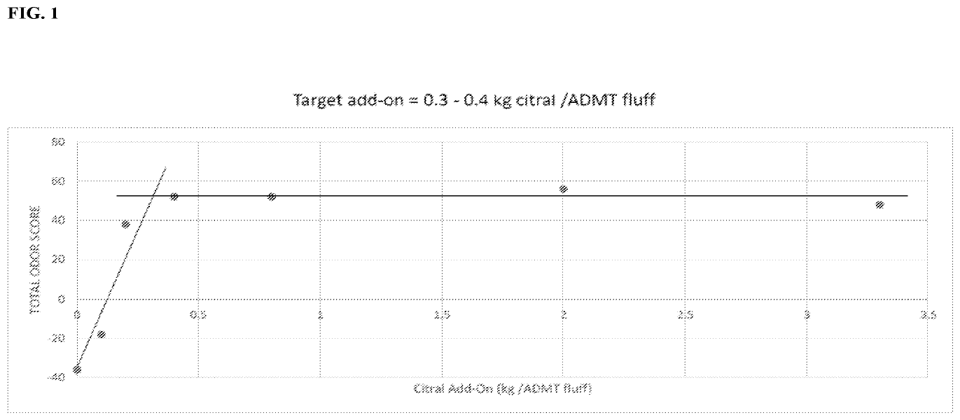

As shown in FIG. 1, odor control performance measured as the TOTAL ODOR SCORE increased with increasing citral add-on up to a value of about 0.3 to 0.4 kg citral per air-dried metric ton of fluff and then remained constant up to a value of 3.3 kg citral per air-dried metric ton of fluff.

The resulting embodiments shown in TABLE 5 were evaluated according to the following method.

### Urine Odor Assessment of OCF Using a Blind, Triangular Forced-Choice Test Method with Expert Panelist

SAMPLE PREPARATION—Individual samples 60 mm in diameter were cut with a die from a sheet of air-laid fluff pulp that had a basis weight of 400 g/cm²±about 5 g/cm² and a density of about 0.06 g/cc. For each odor test, eight samples of a control material and six samples of an unknown, or test material, were made. The samples were placed in cylindrical carbonate or glass jars 70 mm in diameter and 70 mm in height. The volume of a jar was about 270 cm³. Each jar had a plastic screw-on lid. One sample was placed in each jar and dosed with either distilled water or fresh urine and heated in a water bath at 40° C.±5° C. during the test. Dosing was done with a calibrated syringe taking care to evenly distribute the liquid over the sample. Between tests, the jars were washed with an unscented dishwashing liquid.

TEST DESIGN—There were two parts to an odor test. In Part 1, one test sample was dosed with urine and compared to two control samples dosed with distilled water. In Part 2, another test sample that had been dosed with urine was compared to two control samples also dosed with urine. TABLE 6 below provides a comparison.

TABLE 6

| | | Dilution Level | | |
|---|---|---|---|---|
| | time | (g liq/g fluff) | | |
| | (min) | 2.5 | 5 | 10 |
| PART 1 | | | | |
| 2 Control samples with water vs. | 10 | #1 | #2 | #3 |
| 1 test sample with urine | 100 | #7 | #8 | #9 |
| PART 2 | | | | |
| 2 Control samples with urine vs. | 10 | #4 | #5 | #6 |
| 1 test sample with urine | 100 | #10 | #11 | #12 |

Tests #1-#12,
in chronological order, for
assessment of each test
sample.

For each test sample, there are twelve odor comparisons. There are two parts to the test and three levels of urine dosing (i.e. 2.5, 5, and 10 g of liquid per g. of the dry sample) in each part at both 10 and 100 min after dosing. Samples were maintained at a temperature of 40° C.±5° C. during the test. In Part 1 of the test above, for convenience, the same two control samples with 10 g. water/g. fluff were compared to three separate test samples prepared at the three dilution levels of urine. Only two control samples with water are used because the odor of these controls does not change after opening the jar or over time. After the odor test at 10 min., samples are returned to the water bath for an additional 100 min. before the final odor assessments. For Part 2 of the test, two control samples with urine were compared to one test sample with urine and separate samples were used for each comparison. In summary, six samples of the test material were prepared with urine (for Parts 1 and 2 at each of the three dilution levels). Six of the control samples were prepared with urine (for Parts 1 and 2 at each of the three dilution levels) and an additional two control samples were prepared with distilled water (at 10 g water/g fluff) for use in Part 1.

PANELISTS—Each odor assessment was made by having a test administrator remove appropriate jars from the water bath and present them to the panelist blind. The panelist removed the lid of each jar and sniffed the headspace of each jar before recapping. The panelist was instructed to select the one of the three jars that was different from the other two, even if they had to guess. This statistical approach is called “triangular forced-choice”. The panelist would tell the test administrator which sample was different, and whether their selection was a “detection” (i.e., one is confident that their selection was different from the other two) or was a “guess” (i.e., one is not confident that their selection was different from the other two). If the panelist makes a correct selection, and that selection is a “detection”, the test administrator records a value of “6” for that comparison. If the panelist guessed at the selection, a value of “2” was recorded for a correct guess and a value of “0” was recorded for an incorrect guess. TABLE 7 below provides the response key.

TABLE 7
Response Key

| | | |
|---|---|---|
| | 0 | Incorrect guess |
| + unknown better than control | 2 | Correct guess |
| − unknown worse than control | 5 | Incorrect detection |
| | 6 | Correct detection |

Odor values of 2 and 6 are used because sensory responses, such as odor, follow a power law, where the apparent odor intensity increases as a power function of the dosing or odor concentration, i.e., according to the following:

$$I = k {C}^{n} \qquad ( 1 )$$

$$and$$

$${log}_{10} I = n {log}_{10} C + {log}_{10} K \qquad ( 2 )$$

where I is the odor intensity or strength, C is a measure of the mass concentration of odorant, and k and n are constants that are different for every odorant. Equation (2) is linear plotted on a log-log scale. The dilution levels in the test, which double on each step, are related to C, the mass concentration of odorant. Using a log₁₀ transformation forces an equal spread (i.e. 0.301) between the levels of 2.5, 5, and 10 g. liquid/g. fluff. An equal spread is forced between upper and lower values of I, or the Odor Score, in the test. So, log₁₀(2*0.301)=4, i.e., the spread between Odor Score values of 6 and 2.

The panelist also provides the test administrator with an assessment of the pleasantness or unpleasantness of the odor of the test sample relative to that of the odor of the control samples. For example, if the panelist makes a correct selection and is certain of that selection (i.e. it was a “detection”), and the odor of the selected test sample was more pleasant than that of the control samples, the test administrator would record a value of “+6” for that comparison. The test administrator would record a value of “−6” if that test sample was more unpleasant than that of the control samples.

There is one other situation that can occur in the test. If the scented test sample with urine can be confidently identified relative to the control samples with water, but the test sample with urine has no detectable urine odor, the result is recorded as “±6” indicating that either odor is subjectively acceptable. For example, a lemongrass scent with no detectable urine odor can be as acceptable as a sample with no scent at all. The choice depends on the preference of the panelist. In adding up the odor scores, a “±6” is scored as a “+6” because it is as good as an untreated control with distilled water.

Panelists for an odor test can be recruited from the community at large. A person who uses tobacco, is pregnant, or has chronic allergies or asthma or nasal congestion is not a candidate for the odor panel. Panelists must be free of colds and other respiratory ailments for the test and must not chew gum or eat 30 minutes before a test. Panelists must not wear perfume, cologne, aftershave, or scented deodorant on the day of a test. Samples are prepared for each panelist using fresh urine collected from that panelist on the morning of the day of the odor test. Panelists are only required to smell their own urine. Urine is discarded at the end of each day.

ANALYSIS OF ODOR SCORES—Examples below illustrate the calculation and analysis of the Odor Scores. The first example is a test of OCF 31-4 versus untreated RAYFLOC® fluff pulp. That is, RAYFLOC® fluff pulp was the control sample in this odor test.

TABLE 8

| | | Dilution Level | | | | | TOTAL |
|---|---|---|---|---|---|---|---|
| Control (untreated fluff) | time | (g liq/g fluff) | | | | | ODOR |
| Unknown (31-4 OCF sample) | (min) | 2.5 | 5 | 10 | Subtotal | | SCORE |
| PART 1 | | | | | | | |
| 2 untreated fluff samples (control) with water vs. | 10 | ±6 | ±6 | ±2 | +14 | Water (control) vs. | +52 |
| 1 test sample of 31-4 with urine | 100 | ±6 | ±6 | −6 | +6 | Urine (unknown) | |
| | | | | | | ODOR SCORE +20 | |
| PART 2 | | | | | | | |
| 2 untreated fluff samples (control) with urine vs. | 10 | +6 | +6 | +6 | +18 | Urine (control) vs. | |
| 1 test sample of 31-4 with urine | 100 | +6 | +6 | +2 | +14 | Urine (unknown) | |
| | | | | | | ODOR SCORE +32 | |

In Part 1 of the test, at 2.5 g. and 5 g. urine/g. 31-4 OCF sample (10 min.), the Odor Scores were ±6. This indicated that there was no detectable urine odor on the 31-4 sample versus untreated fluff with distilled water. At 10 g. urine/g. OCF sample, a lemongrass scent was barely detectable but there was still no detectable urine odor. At 10 g. urine/g. 31-4 OCF sample and 100 min. there was detectible urine odor on the 31-4 sample, and an Odor Score of “−6” was recorded.

In Part 2 of the test, the odor of the 31-4 OCF sample with urine was detectable (and more pleasant) relative to untreated RAYFLOC® with urine (i.e. “+6” values). At 100 min. and the highest level of urine dosing (i.e. 10 g. urine/g. fiber), the panelist had to guess but guessed correctly that the 31-4 OCF sample was different, and that its odor was more pleasant.

The second example is a test of the 31-4 OCF sample versus itself. That is, the 31-4 OCF sample was the control sample in this odor test.

TABLE 9

| | | Dilution Level | | | | | TOTAL |
|---|---|---|---|---|---|---|---|
| Control (31-4 OCF sample) | time | (g liq/g fluff) | | | | | ODOR |
| Unknown (31-4 OCF sample) | (min) | 2.5 | 5 | 10 | Subtotal | | SCORE |
| PART 1 | | | | | | | |
| 2 test samples of 31-4 with water vs. | 10 | −2 | −6 | −6 | −14 | Water (control) vs. | −32 |
| 1 test sample of 31-4 with urine | 100 | −6 | −6 | −6 | −18 | Urine (unknown) | |
| | | | | | | ODOR SCORE −32 | |
| PART 2 | | | | | | | |
| 2 test samples of 31-4 with urine vs. | 10 | 0 | 0 | 0 | 0 | Urine (control) vs. | |
| 1 test sample of 31-4 with urine | 100 | 0 | 0 | 0 | 0 | Urine (unknown) | |
| | | | | | | ODOR SCORE 0 | |

Tests #1-#12,
in chronological order, for
assessment of each test
sample.

Odor Score values were negative in the first part of the test. This may be a result of it being easier to detect a slight urine odor for a scented 31-4 sample with urine versus a scented 31-4 sample with water than it was to detect a urine odor in the first example for a scented 31-4 sample with urine versus an unscented fluff sample with water. All values in the second part of the test were “0”. All of the samples were the same and the panelist had to guess which one was arbitrarily designated as the test sample. The panelist guessed wrong in all cases, although there was a 33% chance of randomly making a correct choice.

The Odor Score obtained from Part 1 of the test is a comparison of an unknown material with urine versus a control material with distilled water. The Odor Score obtained from Part 2 of the test is a comparison of the same unknown material with urine versus the same control material with urine. The TOTAL ODOR SCORE is the sum of the odor scores from Parts 1 and 2 of the test. Variations of the test can be used with samples containing superabsorbent polymer and samples cut from absorbent cores and absorbent products, as well as using other reference materials for controls. The Odor Scores provide a good semi-quantitative measure of the effectiveness of odor control of the invention.

### Example 4: Odor Control Performance of a Citral/β-cyclodextrin Complex

The following example characterizes the unexpected discovery that the odor control performance of a β-cyclodextrin/citral complex can be improved by forming the complex in solution at pH 12.5 and applying that solution at elevated temperature such that the complex precipitates in situ on the fiber surface at room temperature. The following example also characterizes the unexpected discovery that the odor control performance of the citral: β-cyclodextrin complex exhibited an optimum at a mole ratio of citral: β-cyclodextrin of about 1.5:1.

The following TABLE 10 shows the performance of exemplary citral/β-cyclodextrin embodiments with decreasing add-on of citral (kg citral per air-dried metric ton fluff) using citral: β-cyclodextrin mole ratios of 1.0:1, 1.5:1, and 1.75:1. Additionally, control samples used to generate odor scores were made of untreated RAYFLOC® fluff from Rayonier Performance Fibers, LLC. The odor score was determined by the triangular forced-choice test method discussed above. As seen in the following TABLE 10, the odor control performance was best for a citral: β-cyclodextrin mole ratio of 1.5:1, and was independent of citral add-on over a range of 3.3 to 0.4 kg citral/ADMT fluff pulp at that mole ratio. A citral: β-cyclodextrin mole ratio of 1.75:1 had a slightly detectable scent and color on fluff pulp, indicating that not all of the citral was complexed with the cyclodextrin.

TABLE 10
ODOR SCORE (vs. Rayfloc) AS A FUNCTION OF CITRAL:β-CYCLODEXTRIN MOLE RATIO

| | | | | | | | | | Water | Urine | |
|---|---|---|---|---|---|---|---|---|---|---|---|
| | | | | | | | Add- | | (control) | (control) | |
| | | | | | | | On | | vs. | vs. | |
| | | Citral: | | | | | (kg | | Urine | Urine | |
| | | β-CD | | Mix | Appl'n | | citral/ | TOTAL | (unknown) | (unknown) | |
| Sample | Sample | mole | Solution | Temp | Temp | Appearance | ADMT | ODOR | ODOR | ODOR | Control in |
| No. | ID | ratio | pH | (C.) | (C.) | of Solution | fluff) | SCORE | SCORE | SCORE | Odor Test |
| 1 | 78-2 | 1.0:1 | 9.6 | 83° | 83° | CLEAR | 3.2 | −16 | −36 | +20 | vs. Rayfloc |
| 2 | 78-1 | 1.0:1 | 9.6 | 83° | 83° | CLEAR | 2.2 | −28 | −36 | +8 | vs. Rayfloc |
| 3 | 68-1 | 1.0:1 | 12.5 | 85° | 85° | CLEAR | 2.0 | +16 | −12 | +28 | vs. Rayfloc |
| 4 | 53-1 | 1.0:1 | 12.5 | 87° | 87° | CLEAR | 1.4 | −26 | −36 | +10 | vs. Rayfloc |
| 5 | 25-1 | 1.5:1 | 12.5 | 86° | 86° | CLEAR | 3.3 | +48 | +12 | +36 | vs. Rayfloc |
| 6 | 31-1 | 1.5:1 | 12.5 | 86° | 86° | CLEAR | 2.0 | +56 | +24 | +32 | vs. Rayfloc |
| 7 | 31-4 | 1.5:1 | 12.5 | 86° | 86° | CLEAR | 0.8 | +52 | +20 | +32 | vs. Rayfloc |
| 8 | 31-5 | 1.5:1 | 12.5 | 86° | 86° | CLEAR | 0.4 | +52 | +24 | +28 | vs. Rayfloc |
| 9 | 37-1 | 1.5:1 | 12.5 | 86° | 86° | CLEAR | 0.2 | +38 | +20 | +18 | vs. Rayfloc |
| 10 | 37-2 | 1.5:1 | 12.5 | 86° | 86° | CLEAR | 0.1 | −18 | −36 | +18 | vs. Rayfloc |
| 11 | 72-1 | 1.75:1 | 12.5 | 85° | 85° | CLEAR | 0.8 | +60 | +24 | +36 | vs. Rayfloc |
| 12 | 60-1 | 1.75:1 | 12.5 | 86° | 86° | CLEAR | 0.4 | −14 | −24 | +10 | vs. Rayfloc |

The best odor control performance was achieved by using a citral: β-cyclodextrin mole ratio of 1.5:1 and a solution pH of 12.5. TABLE 11 below shows the performance of exemplary citral/β-cyclodextrin embodiments made with a citral: β-cyclodextrin mole ratio of 1.5:1 as a function of solution pH, mix temperature, and application temperature. The first group of three samples was made with a solution that was mixed and applied at an elevated temperature of 86° C. For the second group, the solution was mixed at elevated temperature but allowed to cool and applied at room temperature. The solution was mixed and applied at room temperature for the third group. Odor scores increased (i.e., performance improved) with increasing pH for each group. There was no significant difference between groups as a result of normal variation in the odor test scores.

TABLE 11
ODOR SCORES (vs. 31-4) AS A FUNCTION OF pH, MIX TEMP AND
APPLICATION TEMP AT 1.5:1 Citral:CD MOLE RATIO

| | | | | | | | | | Water | Urine | |
|---|---|---|---|---|---|---|---|---|---|---|---|
| | | | | | | | Active | | (control) | (control) | |
| | | | | | | | Add- | | vs. | vs. | |
| | | Citral: | | | | | On (kg | | Urine | Urine | Control |
| | | β-CD | | Mix | Appl'n | Appearance of | citral/ | TOTAL | (unknown) | (unknown) | in |
| Sample | Sample | mole | Solution | Temp | Temp | Solution at | ADMT | ODOR | ODOR | ODOR | Odor |
| No. | ID | ratio | pH | (C.) | (C.) | Appl'n Temp | fluff) | SCORE | SCORE | SCORE | Test |
| 1 | 31-4 | 1.5:1 | 12.5 | 86° | 86° | CLEAR | 0.8 | −32 | −32 | 0 | vs. 31-4 |
| 2 | 58-1 | 1.5:1 | 9.6 | 86° | 86° | CLEAR | 0.8 | −46 | −36 | −10 | vs. 31-4 |
| 3 | 55-1 | 1.5:1 | 6.5 | 86° | 86° | CLEAR | 0.8 | −54 | −36 | −18 | vs. 31-4 |
| 4 | No | 1.5:1 | 12.5 | 86° | 20° | ORANGE- | 0.8 | — | — | — | — |
| | sample | | | | | BROWN ON | | | | | |
| | made | | | | | COOLING | | | | | |
| 5 | 45-1 | 1.5:1 | 9.6 | 86° | 20° | MILKY WHITE | 0.8 | −36 | −32 | −4 | vs. 31-4 |
| 6 | 43-1 | 1.5:1 | 6.5 | 86° | 20° | MILKY WHITE | 0.8 | −54 | −36 | −18 | vs. 31-4 |
| 7 | 51-3 | 1.5:1 | 12.5 | 20° | 20° | MILKY WHITE | 0.8 | −22 | −30 | +8 | vs. 31-4 |
| 8 | 62-1 | 1.5:1 | 9.6 | 20° | 20° | MILKY WHITE | 0.8 | −32 | −36 | +4 | vs. 31-4 |
| 9 | 67-1 | 1.5:1 | 6.5 | 20° | 20° | MILKY WHITE | 0.8 | −66 | −36 | −30 | vs. 31-4 |

The control sample used to generate the odor scores in TABLE 11 was a 31-4 sample as used in Example 3. A 31-4 sample was also tested as Sample No. 1 in TABLE 11 above. All of the water (i.e., control) versus urine odor scores were negative because it is easier to detect a slight difference in urine malodor between similarly scented samples than it is to detect a slight urine malodor in a scented sample versus an unscented sample such as RAYFLOC® fluff treated with water. The 31-4 sample was favored because the complex formed a clear, homogeneous solution at an elevated temperature of about 86° C. This step did not improve the odor control performance of the complex. However, it greatly improved the ability to handle the solution and uniformly apply it to a substrate surface. A solution of a β-cyclodextrin/citral complex sprayed on fluff pulp at an elevated temperature of about 86° C. would precipitate in situ on the fiber surface as it cooled to room temperature.

Turbidity is an indicator of the solubility of the complex in solution. A soluble complex forms a clear, water-white solution. An insoluble complex forms an opaque, milky-white dispersion that can settle over time. A milky-white dispersion is formed if a solution at 86° C. cools to room temperature or if no heat is used in the mixing or application steps. A simulated laboratory kiss roll method was used to apply the milky-white dispersions of the complex to fluff pulp. A known amount of the milky-white dispersion was applied to a glass surface and a fluff pulp sheet was immediately placed in contact with the glass surface before the dispersion dried. Wet pick-up of the dispersion was estimated by immediately weighing the sheet of fluff pulp.

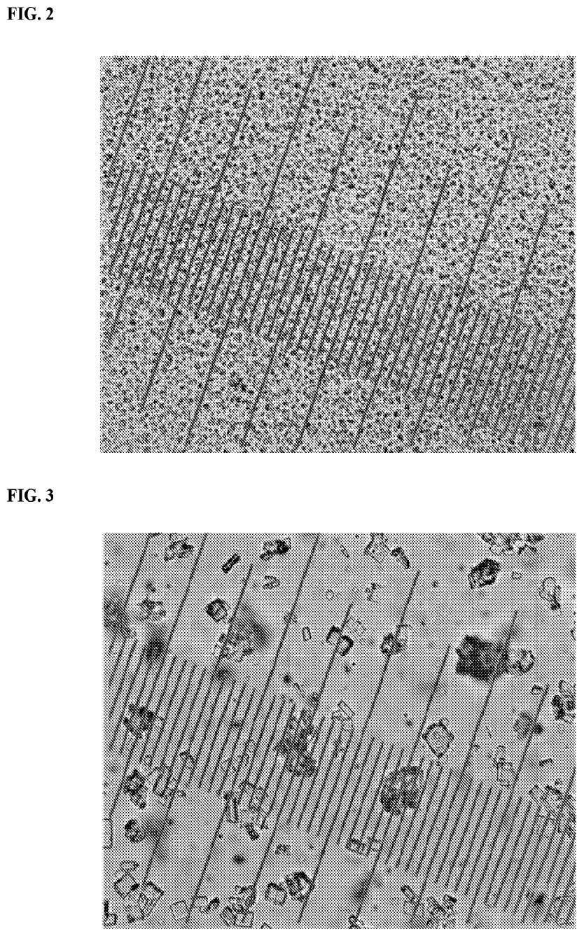

Without being bound to a singular and complete understanding of the physical-chemical modifications imparted by the interaction of a citral/β-cyclodextrin complex with solution pH, the inventors believe that the unique physical form of the precipitate formed on a solid cellulosic surface may lead to the unexpected performance enhancement in odor control for samples made using an elevated solution pH of 12.5. FIG. 2 and FIG. 3 are photomicrographs below showing the particle size of citral/β-cyclodextrin complexes precipitated from 86° C. glycerol/water solutions at pH 12.5 and 9.6 after the solutions cooled to room temperature.

Solid particles of the complex precipitated from a 12.5 pH solution were uniform in dimension and about 50 μm in diameter. Particles precipitated from the solution at pH 9.6 had dimensions in the range of 100-400 μm. It was an unexpected discovery to learn that the odor control performance of a β-cyclodextrin/citral complex could be improved by forming the complex in a glycerol/water solution at a citral: β-cyclodextrin mole ratio of about 1.5:1 and at an elevated pH of about 12.5.

### Example 5: Substrate Preparation

A sample was prepared using an 85/15 ratio of glycerol/water and a pH adjustment of 9.6using β-cyclodextrin from Acros Organics and citral, a volatile lemongrass-scented essential oil, from Perfumer's Apprentice. The solution was mixed and applied at a temperature of 86° C. At the application temperature of 86° C., the components formed a clear, water-white solution. The citral to β-cyclodextrin mole ratio was 1.0. The amount of citral added on was 2.4 kg of citral per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the substrate was 1.0 wt %. This was checked using a METTLER TOLEDO® Moisture Analyzer where the treated sample had a moisture content of 6.29%±0.18% and the untreated Rayfloc control had a moisture content of 5.33%±0.39%. An experimental value of 0.96% was obtained for the moisture increase of the sample. Because the moisture increase was less than about 1 wt % or less, no drying step was required. The overall odor score was −28 as determined by the blind, triangular forced-choice test method with an expert panel discussed above. This sample did not provide good odor control performance even for this relatively high amount of citral add-on. Citral add-on was 2.4 kg of citral per air-dried metric ton (“ADMT”) of fluff. Odor control performance was poor because the citral: β-cyclodextrin mole ratio was less than 1.5:1 and the solution pH was less than pH 12. Specifications and odor score measurements for Example 5 are given in TABLE 12 and TABLE 13.

TABLE 12
SPECIFICATION

| | | | | | | | | | CD | Citral | Glycerol | |
|---|---|---|---|---|---|---|---|---|---|---|---|---|
| | | | | | | | Solution | Solids | Add-On | Add-On | Add-On (kg | Fluff |
| | | Solution | | | | | Add-on | Add-On | (kg CD/ | (kg Citral/ | glycerol/ | Moisture |
| | | | Liquid | Soln Wt. | T | | (g soln/ | (g solids/ | ADMT | ADMT | ADMT | Increase |
| Sample | Description | Component | Conc | Fraction | (° C.) | pH | g fluff) | g fluff) | fluff) | fluff) | fluff) | (%) |
| Example | β-CD/ | glycerol | 0.85 | 0.658 | 86 | 9.6 | 0.088 | 0.020 | 17.6 | 2.4 | 58.2 | 1.0% |
| | Citral | water | 0.15 | 0.116 | | | | | | | | |
| | complex | Subtotal | | 0.774 | | | | | | | | |
| | | β-Cyclodextrin | | 0.199 | | | | | | | | |
| | | Citral | | 0.027 | | | | | | | | |
| | | Total | | 1.00 | | | | | | | | |

Mole Ratio (mol CD/mol Citral) = 1.0

| | Solution Preparation: | | |
|---|---|---|---|
| MW (g/mol) | | g used | % |

| | | | | | |
|---|---|---|---|---|---|
| 1135 | CD | 12.9 | 19.9% | 22.6% | Total 14.6 |
| 152.2 | Citral | 1.73 | 2.7% | | |
| | Soln | 50.0 | 77.4% | | |
| | Total | 64.6 | 100% | | |

TABLE 13
ODOR EVALUATION

| Triangular Forced-Choice | | | | Liquid Dosing Level | | | Odor | | |
|---|---|---|---|---|---|---|---|---|---|
| Odor Evaluation | | | time | (g liq/g fluff) | | | Score | | |
| Test | 2 Controls | 1 Unknown | (min) | 2.5 | 5 | 10 | Summary | | |
| 1 | Rayfloc/water | OCF/urine | 10 | 6− | 6− | 6− | −18 | −36 | −28 |
| | | | 100 | 6− | 6− | 6− | −18 | | |
| 2 | Rayfloc/urine | OCF/urine | 10 | 2+ | 2+ | 0 | +4 | +8 | |
| | | | 100 | 2+ | 2+ | 0 | +4 | | |

### Example 6: Substrate Preparation

A sample was prepared using an 85/15 ratio of glycerol/water and a pH adjustment of 12.5 using β-cyclodextrin from Acros Organics and citral from Perfumer's Apprentice. The solution was mixed and applied at a temperature of 86° C. At the application temperature of 86° C., the components formed a clear, water-white solution. The citral to β-cyclodexrin mole ratio was 1.0. The amount of citral added on was 2.4 kg of citral per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the substrate was 1.0 wt. %. Because the moisture increase was less than about 1 wt. % or less, no drying step was required. The overall odor score was +16 as determined by the blind, triangular forced-choice test method with an expert panel discussed above. This sample provided better odor control than the sample in Example 5 because of an increase in solution pH from 9.6 to 12.5. Specifications and odor score measurements for Example 6 are given in TABLE 14 and TABLE 15.

TABLE 14
SPECIFICATION

| | | | | | | | | | CD | Citral | Glycerol | |
|---|---|---|---|---|---|---|---|---|---|---|---|---|
| | | | | | | | Solution | Solids | Add-on | Add-On | Add-On (kg | Fluff |
| | | Solution | | | | | Add-on | Add-On | (kg CD/ | (kg Citral/ | glycerol/ | Moisture |
| | | | Liquid | Soln Wt. | T | | (g soln/ | (g solids/ | ADMT | ADMT | ADMT | Increase |
| Sample | Description | Component | Conc | Fraction | (° C.) | pH | g fluff) | g fluff) | fluff) | fluff) | fluff) | (%) |
| Example | β-CD/ | glycerol | 0.85 | 0.638 | 86 | 12.5 | 0.080 | 0.020 | 17.6 | 2.4 | 51.2 | 0.9% |
| | Citral | water | 0.15 | 0.113 | | | | | | | | |
| | complex | Subtotal | | 0.751 | | | | | | | | |
| | | β-Cyclodextrin | | 0.220 | | | | | | | | |
| | | Citral | | 0.029 | | | | | | | | |
| | | Total | | 1.00 | | | | | | | | |

Mole Ratio (mol CD/mol Citral) = 1.0

Solution Preparation:

| | | |
|---|---|---|
| MW (g/mol) | g used | % |

| | | | | | |
|---|---|---|---|---|---|
| 1135 | CD | 14.6 | 22.0% | 24.9% | Total 16.6 |
| 152.2 | Citral | 1.96 | 2.9% | | |
| | Soln | 50.0 | 75.1% | | |
| | Total | 66.6 | 100% | | |

TABLE 15
ODOR EVALUATION

| Triangular Forced-Choice | | | | Liquid Dosing Level | | | Odor | | |
|---|---|---|---|---|---|---|---|---|---|
| Odor Evaluation | | | time | (g liq/g fluff) | | | Score | | |
| Test | 2 Controls | 1 Unknown | (min) | 2.5 | 5 | 10 | Summary | | |
| 1 | Rayfloc/water | OCF/urine | 10 | 6± | 6− | 6− | −6 | −12 | +16 |
| | | | 100 | 6± | 6− | 6− | −6 | | |
| 2 | Rayfloc/urine | OCF/urine | 10 | 6+ | 6+ | 6+ | +18 | +28 | |
| | | | 100 | 6+ | 2+ | 2+ | +10 | | |

### Example 7: Substrate Preparation

A sample was prepared using an 85/15 ratio of glycerol/water and a pH adjustment of 12.5 using β-cyclodextrin from Acros Organics and citral from Perfumer's Apprentice. The solution was mixed and applied at a temperature of 86° C. At the application temperature of 86° C., the components formed a clear, water-white solution. The citral to β-cyclodextrin mole ratio was 1.5. The amount of citral added on was 2.0 kg of citral per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the substrate was 0.6 wt. %. Because the moisture increase was less than about 1 wt. % or less, no drying step was required. The overall odor score was +56 as determined by the blind, triangular forced-choice test method with an expert panel discussed above. This sample provided very good odor control performance as a result of a citral to β-ctyclodextrin mole ratio of 1.5:1 and a solution pH of 12.5. Specifications and odor score measurements for Example 7 are given in TABLE 16 and TABLE 17.

TABLE 16
SPECIFICATION

| | | | | | | | | Solids | CD | Citral | Glycerol | |
|---|---|---|---|---|---|---|---|---|---|---|---|---|
| | | | | | | | Solution | Add- | Add- | Add- | Add-On (kg | |
| | | Solution | | | | | Add-on | On | On | On | glycerol/ | Fluff |
| | | | Liquid | Soln Wt. | T | | (g | (g | (kg | (kg | ADMT | Moisture |
| Sample | Description | Component | Conc | Fraction | (° C.) | pH | soln | solids | CD | Citral | fluff) | Increase |
| Example | β-CD/ | glycerol | 0.85 | 0.666 | 86 | 12.5 | 0.055 | 0.012 | 9.9 | 2.0 | 36.8 | 0.6% |
| | Citral | water | 0.15 | 0.118 | | | | | | | | |
| | complex | Subtotal | | 0.784 | | | | | | | | |
| | | β-Cyclodextrin | | 0.180 | | | | | | | | |
| | | Citral | | 0.036 | | | | | | | | |
| | | Total | | 1.00 | | | | | | | | |

Mole Ratio (mol CD/mol Citral) = 1.5

Solution Preparation:

| MW (g/mol) | | g used | % | | |
|---|---|---|---|---|---|
| 1135 | CD | 11.5 | 18.0% | 21.6% | Total 13.8 |
| 152.2 | Citral | 2.31 | 3.6% | | |
| | Soln | 50.0 | 78.4% | | |
| | Total | 63.8 | 100% | | |

TABLE 17
ODOR EVALUATION

| Triangular Forced-Choice | | | | Liquid Dosing Level | | | Odor | | |
|---|---|---|---|---|---|---|---|---|---|
| Odor Evaluation | | | time | (g liq/g fluff) | | | Score | | |
| Test | 2 Controls | 1 Unknown | (min) | 2.5 | 5 | 10 | Summary | | |
| 1 | Rayfloc/water | OCF/urine | 10 | 6± | 6± | 6± | +18 | +24 | +56 |
| | | | 100 | 6± | 6± | 6− | +6 | | |
| 2 | Rayfloc/urine | OCF/urine | 10 | 6+ | 6+ | 6+ | +18 | +32 | |
| | | | 100 | 6+ | 6+ | 2+ | +14 | | |

### Example 8: Substrate Preparation

A sample was prepared using an 85/15 ratio of glycerol/water and a pH adjustment of 12.5 using β-cyclodextrin from Acros Organics and citral from Perfumer's Apprentice. The solution was mixed and applied at a temperature of 86° C. At the application temperature of 86° C., the components formed a clear, water-white solution. The citral to β-cyclodextrin mole ratio was 1.5. The amount of citral added on was 0.4 kg of citral per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the substrate was 0.1 wt. %. Because the moisture increase was less than about 1 wt. % or less, no drying step was required. The overall odor score was +52 as determined by the blind, triangular forced-choice test method with an expert panel discussed above. At an add-on of only 0.4 kg citral/ADMT fluff pulp, this sample provided odor control performance as good as that for a sample made with an add on of 2.0 kg citral/ADMT fluff pulp (i.e. Example 7). Specifications and odor score measurements for Example 8 are given in TABLE 18 and TABLE 19.

TABLE 18
SPECIFICATION

| | | | | | | | | Solids | CD | Citral | Glycerol | |
|---|---|---|---|---|---|---|---|---|---|---|---|---|
| | | | | | | | | Add- | Add- | Add- | Add-On | |
| | | Solution | | | | | Solution | On | On | On | (kg glycerol/ | Fluff |
| | | | Liquid | Soln Wt. | T | | Add-on | (g | (kg | (kg | ADMT | Moisture |
| Sample | Description | Component | Conc | Fraction | (° C.) | pH | (g soln | solids | CD | Citral | fluff) | Increase |
| Example | β-CD/Citral | glycerol | 0.85 | 0.666 | 86 | 12.5 | 0.011 | 0.002 | 2.0 | 0.40 | 7.4 | 0.1% |
| | complex | water | 0.15 | 0.118 | | | | | | | | |
| | | Subtotal | | 0.784 | | | | | | | | |
| | | β-Cyclodextrin | | 0.180 | | | | | | | | |
| | | Citral | | 0.036 | | | | | | | | |
| | | Total | | 1.00 | | | | | | | | |

Mole Ratio (mol CD/mol Citral) = 1.5

| | Solution Preparation: | | |
|---|---|---|---|
| MW (g/mol) | | g used | % |

| 1135 | CD | 11.5 | 18.0% | 21.6% | Total 13.8 |
|---|---|---|---|---|---|
| 152.2 | Citral | 2.31 | 3.6% | | |
| | Soln | 50.0 | 78.4% | | |
| | Total | 63.8 | 100% | | |

TABLE 19
ODOR EVALUATION

| Triangular Forced-Choice | | | | | | | | | |
|---|---|---|---|---|---|---|---|---|---|
| Odor Evaluation | | | time | Liquid Dosing Level | | | Odor | | |
| Test | 2 Controls | 1 Unknown | (min) | 2.5 | 5 | 10 | Score | | |
| 1 | Rayfloc/water | OCF/urine | 10 | 6± | 6± | 6± | +18 | +24 | +52 |
| | | | 100 | 6± | 6± | 6− | +6 | | |
| 2 | Rayfloc/urine | OCF/urine | 10 | 6+ | 6+ | 2+ | +14 | +28 | |
| | | | 100 | 2+ | 6+ | 6+ | +14 | | |

### Example 9: Substrate Preparation

A sample was prepared using a 100% water (i.e. a 0/100 ratio of glycerol/water) and a pH adjustment of 12.5 using β-cyclodextrin from Acros Organics and citral from Perfumer's Apprentice. The solution was mixed and applied at a temperature of 86° C. At the application temperature of 86° C., the components formed a relatively stable, milky-white dispersion. The citral to β-cyclodextrin mole ratio was 1.5. The amount of citral add-on was 0.4 kg of citral per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the substrate was 1.1 wt. %. Because the moisture increase was about 1 wt. %, no drying step was required. The overall odor score was +36 as determined by the blind, triangular forced-choice test method with an expert panel discussed above. The odor control performance of this reduced-cost, 100% water solution sample was good—almost as good as the sample made in Example 8 with the 85/15 glycerol/water solution. The mixture formed a dispersion after the addition of the citral and formation of the complex at 86° C., but when the concentration of cyclodextrin and citral in the solution was less than about 20 wt. %, the size of the precipitated particles was very small, odor control performance was good and the dispersion could be sprayed easily onto the substrate. Specifications and odor score measurements for Example 10 are given in TABLE 20 and TABLE 21.

TABLE 20
SPECIFICATION

| | | | | | | | | | CD | Citral | Glycerol | |
|---|---|---|---|---|---|---|---|---|---|---|---|---|
| | | | | | | | Solution | Solids | Add-On | Add-On | Add-On (kg | Fluff |
| | | Solution | | | | | Add-on | Add-On | (kg CD/ | (kg Citral/ | glycerol/ | Moisture |
| | | | Liquid | Soln Wt. | T | | (g soln/ | (g solids/ | ADMT | ADMT | ADMT | Increase |
| Sample | Description | Component | Conc | Fraction | (° C.) | pH | g fluff) | g fluff) | fluff) | fluff) | fluff) | (%) |
| Example | β-CD/ | glycerol | 0.00 | 0.000 | 86 | 12.5 | 0.013 | 0.002 | 2.0 | 0.40 | 0.0 | 1.1% |
| | Citral | water | 1.00 | 0.822 | | | | | | | | |
| | complex | Subtotal | | 0.822 | | | | | | | | |
| | | β-Cyclodextrin | | 0.148 | | | | | | | | |
| | | Citral | | 0.030 | | | | | | | | |
| | | Total | | 1.00 | | | | | | | | |

Mole Ratio (mol CD/mol Citral) = 1.5

Solution Preparation:

| MW (g/mol) | | g used | % | | |
|---|---|---|---|---|---|
| 1135 | CD | 9.0 | 14.8% | 17.8% | Total 10.8 |
| 152.2 | Citral | 1.81 | 3.0% | | |
| | Soln | 50.0 | 82.2% | | |

TABLE 21
ODOR EVALUATION

| Triangular Forced-Choice | | | | Liquid Dosing Level | | | Odor | | |
|---|---|---|---|---|---|---|---|---|---|
| Odor Evaluation | | | time | (g liq/g fluff) | | | Score | | |
| Test | 2 Controls | 1 Unknown | (min) | 2.5 | 5 | 10 | Summary | | |
| 1 | Rayfloc/water | OCF/urine | 10 | 6± | 6± | 6− | +6 | 0 | +36 |
| | | | 100 | 6± | 6− | 6− | −6 | | |
| 2 | Rayfloc/urine | OCF/urine | 10 | 6+ | 6+ | 6+ | +18 | +36 | |
| | | | 100 | 6+ | 6+ | 6+ | +18 | | |

### Conclusion and Summary of Examples 5-9

A summary of Examples 5 to 9 is given in TABLE 22 below. At a glycerol/water ratio of 85/15, and citral add on in the range of 2.0-2.4 kg citral/ADMT fluff pulp, an increase in the citral: cyclodextrin mole ratio from 1:1 to 1.5:1 increased the odor control performance (as indicated by increasing TOTAL ODOR SCORE) by about as much as an increase in solution pH from 9.6 to 12.5. The unadjusted pH of the solution was about 6.5. Using a lower add-on of the same solution to provide a reduced citral add-on of 0.4 kg citral/ADMT fluff pulp provided about the same level of very good performance (i.e. +52), again at a citral: cyclodextrin mole ratio of 1.5:1 and a pH of 12.5. Examples 5 to 8 had clear, water-white solutions at the mixing and application temperature of 86° C. The complex precipitated on the surface of the cellulose fibers as the solution and treated fibers cooled to room temperature.

TABLE 22

| | | | Citral/CD | Citral | | | Moisture | Total Odor |
|---|---|---|---|---|---|---|---|---|
| | | Citral: | Complex | Add-on | | Mixing & | Increase | Control |
| | Glycerol/ | CD | Solution | (kg Citral/ | | Application | of | Score vs. |
| | Water | Mole | Conc. | ADMT | | Temp | Substrate | Rayfloc |
| Example | Ratio | Ratio | (wt %) | fluff pulp) | pH | (° C.) | (wt %) | fluff pulp |
| 5 | 85/15 | 1:1 | 22.6% | 2.4 | 9.6 | 86° C. | 1.0% | −28 |
| 6 | 85/15 | 1:1 | 24.9% | 2.4 | 12.5 | 86° C. | 0.9% | +16 |
| 7 | 85/15 | 1.5:1 | 21.6% | 2.0 | 12.5 | 86° C. | 0.6% | +56 |
| 8 | 85/15 | 1.5:1 | 21.6% | 0.4 | 12.5 | 86° C. | 0.1% | +52 |
| 9 | 0/100 | 1.5:1 | 17.8% | 0.4 | 12.5 | 86° C. | 1.1% | +36 |

It was an unexpected discovery to learn that the odor control performance of a β-cyclodextrin/citral complex could be greatly improved by forming the complex in a glycerol/water solution at a citral: β-cyclodextrin mole ratio of about 1.5:1 and at an elevated pH of about 12.5.

The exact physical dimensions and morphology of the cyclodextrin complex that precipitated in situ on the solid fiber surface have not been characterized. However, without being bound to a singular theory, the inventors believe that the particle size of the complex precipitated in solution, as the solution cools to room temperature, is a measure of the morphology of the complex on a substrate surface. The mean size of solid particles of the complex precipitated from a solution at an elevated temperature at pH 12.5 is in the range of 50 μm or less. A solution pH of about 12.5 or greater should be used for good odor control performance. Odor control performance is poorer when a solution pH of 9.6 or less is used to treat a substrate. The mean size of solid particles of complex precipitated from a solution at an elevated temperature at pH 9.6 or less is in the range of 100-400 μm. The particle size is believed to be controlled by the pH of the solution, inter alia.

The above-discussed results were unanticipated and unexpected. There is no known discussion of the effect of pH on the odor control performance of cyclodextrin complexes in the prior art. The effect may be consistent with the inventors' additional discovery that the complex precipitates into very fine particles when it is precipitated from a solution with a pH of around 12.5. Odor control performance of a complex deposited from solution may be related to its solid phase morphology on the substrate surface. Hydroxyl groups on the outer surface of a cyclodextrin molecule start to deprotonate at about pH 12 (i.e. pKa range about 12.1-13.5). It is known that cyclodextrins and their complexes undergo self-assembly in solution to form aggregate structures. This aggregate formation might be affected by a surface charge related to deprotonation, and in turn, affect the size of precipitated particles and the morphology of complexes deposited on a substrate surface. Others, such as in U.S. Pat. No. 5,429,628, have claimed that small cyclodextrin particles improve the release and speed of release of active.

Particles of less than about 12 microns were preferred for providing a quick release of active compounds when the complexes were wetted. U.S. Pat. No. 5,429,628, however, achieved this reduction in particle size via grinding and pulverizing in a fluid energy mill, or by agitation or sonication. The inventors are unaware of the use of solution pH to adjust the particle size of cyclodextrin complexes, as disclosed herein.

The inventors also unexpectedly discovered that the odor control performance can be sensitive to the molar ratio of citral to cyclodextrin in the complex. U.S. Pat. No. 5,429,628 and WO 98/26808 indicate that, in general, perfume/cyclodextrin complexes have a molar ratio of perfume to cyclodextrin of about 1:1, although sometimes greater, but typically the loading of perfume to cyclodextrin is about 7 wt. % to 12 wt. %. There are practical limitations to the loading of perfume in cyclodextrin complexes, but the inventors are unaware of any disclosure about how loading affects odor control performance. For citral/β-cyclodextrin complexes, a molar ratio of citral to β-cyclodextrin of 1:1 would correspond to a loading of 11.8 wt. %, as generally disclosed by U.S. Pat. No. 5,429,628. However, the molar ratio of citral to β-cyclodextrin of 1.5:1 would correspond to a loading of 16.7 wt. %. Taking into account a moisture content of β-cyclodextrin of 10 wt. %, the molar ratio of citral to dry β-cyclodextrin of 1.5:1 would correspond to a loading of 18.3 wt. %. That is, such ranges of citral loading are well outside the range taught in U.S. Pat. No. 5,429,628. The inventors are unaware of a technical explanation for the disclosed higher range of citral loading, which may be facilitated by the high solution pH in which the complex is formed. Even though it may be possible that the actual quantitative values could be different for the loading of different essential oils, the inventors expect that the requirements for achieving excellent odor control include a minimum loading value of essential oil to cyclodextrin and a solution pH above about 12.0, as a common feature of these systems.

The complexes in Examples 5 to 9 form a clear, homogeneous solution at an elevated temperature of about 86° C. This step did not improve the odor control performance of the complex. However, it greatly improved the ability to handle the solution and uniformly apply it to a substrate surface. A solution of a β-cyclodextrin/citral complex sprayed on fluff pulp at an elevated temperature of about 86° C. would precipitate in situ on the fiber surface as it cooled to room temperature. Solution concentrations of the citral/cyclodextrin complexes were in the range of 21.6 wt. %-24.9 wt. %. The solution concentration of the complex can reduce the solution add-on to keep the moisture increase of the substrate below about 1 wt. %. However, the ratio of odor-controlling compound to cyclodextrin is also supportive for imparting good odor control performance to a substrate. For citral, the optimum molar ratio of citral to β-cyclodextrin is about 1.5-1.75 moles of citral to 1.0 mole of β-cyclodextrin. At molar ratios of about 1:1 and less, odor control performance is reduced (at equal add-ons of citral to substrate). At molar ratios of 1.75:1 and greater, volatile, uncomplexed citral imparts unnecessary color and scent to the substrate. Volatile, and costly, scented essential oils used in absorbent products tend to be lost prematurely. It is desirable to have the essential oils contained in a stable complex and released only when in contact with aqueous body fluids.

The ratio of glycerol/water also plays a role in the solution chemistry discussed above. For example, samples made with higher ratios of water (e.g., 50% glycerol/50% water) do not perform as well as those made with the 85% glycerol/15% water ratio, even when they are made at the preferred 1.5 moles citral: mole cyclodextrin and pH 12.5.

However, it is noteworthy that Example 9 provided almost as good odor control performance using a 100% water solution and was capable of delivering 0.4 kg citral/ADMT fluff pulp with a moisture increase of only 1.1%. This unexpected, practical discovery provides good odor control performance at much reduced raw material cost. In this embodiment, for good odor control performance from a 100% water solution, conditions must be right for the formation of a fine precipitate upon the addition of citral to a β-cyclodextrin/water solution. A solution concentration at 86° C. and pH 12.5 for the complex is lower-in the range of about 18 wt. % or less. At higher concentrations, an undesirably coarse precipitate forms and odor control performance deteriorates.

### Examples 10 to 17

### Triangular Forced-Choice Odor Evaluation Test Protocol Used in Examples 10 to 17

Odor scores were derived from comparisons of a test sample relative to a reference, or control, sample. Odor scores are not absolute values. Both test and control samples were dosed with fresh human urine that was aged for 8 to 12 hours at room temperature for increased malodor. The odor scores in Examples 10 to 17 below were the sum of four head-to-head comparisons of a test sample versus a reference sample at two levels of urine dosing (i.e., 5 g. and 10 g. of urine per g. of fluff) and two times after dosing (i.e. 10 min. and 100 min.). The range of scoring for this comparison was −24 to +24. In this Triangular Forced-Choice Odor Evaluation Test, the panelist selects one of three samples that has a different scent than the other two. A selection must be made by the panelist—there is no possibility of “no difference.” A guess is when the panelist is unsure of their selection. A detection is when the panelist is sure of their selection. The grading scale is provided in TABLE 23 below:

TABLE 23

| Odor | |
|---|---|
| Score | |
| Key | |
| 0 | Incorrect guess |
| +2 | Correct guess (UNKNOWN better than CONTROL) |
| −2 | Correct guess (UNKNOWN worse than CONTROL) |
| +6 | Correct detection (UNKNOWN better than CONTROL) |
| −6 | Correct detection (UNKNOWN worse than CONTROL) |
| ±2 | Correct guess (preference determines whether better or worse) |
| ±6 | Correct detection (preference determines whether better or worse) |

The ±2 and ±6 scores are counted as a +2 and +6, respectively

The samples in Examples 10 to 17 were also tested versus conventional fluff for the lower urine dosing level at two times (i.e., 5.0 g. of urine per g. of fluff at 10 and 100 min.). The range of scoring for this comparison was −12 to +12. Except for the conventional fluff samples in Example 17, all test and control samples in Examples 10 to 17 scored a perfect +12 versus the conventional fluff. This indicated that all control and test samples in Examples 10 to 17 masked urine better than conventional fluff.

### Example 10: Comparison of a Citral/Coconut Fragrance Blend to Citral Alone

A sample similar to that described in Example 9 was prepared using 100% water (i.e. a 0/100 ratio of glycerol/water) and a pH adjustment of 12.5 using β-cyclodextrin from Wacker Chemical Co. and citral from Advanced Biotech. The solution was prepared at a citral to β-cyclodextrin mole ratio of 1.6 instead of 1.5, not a meaningful difference, and mixed and applied at a temperature of 86° C. The amount of citral add-on was 0.4 kg of citral per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the substrate was 1.0 wt. %. Because the moisture increase was about 1 wt. %, no drying step was required. This sample, identified as 4-1, was used as the control sample in a Triangular Forced-Choice Odor Evaluation versus a test sample, identified as 12-1, prepared using an equimolar blend of citral and coconut fragrance oil at the same total essential oil to β-cyclodextrin mole ratio of 1.6 and the same mixing and application temperature of 86° C. The total essential oil add-on for the equimolar blend of citral and coconut fragrance oil was increased to 0.46 kg per air-dried metric ton (“ADMT”) of fluff to keep the samples at a comparable raw material cost. The coconut fragrance oil, supplied by Perfumer's Apprentice, was a blend of several essential oils, which is shown in TABLE 24 below.

TABLE 24

| | CAS# | Conc. | Molecular |
|---|---|---|---|
| | Ingredient | Range | Weight (g/mol) |
| benzyl benzoate | 120-51-4 | 40-50 | 212.2 |
| gamma-nonalactone | 104-61-0 | 30-40 | 156.2 |
| coumarin | 91-64-5 | 10-20 | 146.1 |
| vanillin | 121-33-5 | 2-5 | 152.2 |
| gamma-decalactone | 706-14-9 | 2-5 | 170.3 |

The weighted average molecular weight of the coconut fragrance oil was about 180.2 g/mole. Sample 12-1 was also made using β-cyclodextrin from Wacker Chemical Co. and citral from Advanced Biotech.

The odor score of Test Sample 12-1 containing the citral/coconut fragrance blend versus Control Sample 4-1 was +24, out of a possible range of −24 to +24, as determined in the blind, triangular forced-choice test discussed above. This showed that an equimolar blend of citral and coconut fragrance oil, at a somewhat higher add-on level but at comparable raw material cost, improved the odor-control performance over that of a sample made with citral alone. Specifications for Control Sample 4-1 and Test Sample 12-1 are given in TABLE 25 (Control Sample 4-1) and TABLE 26 (Test Sample 12-1). Odor score measurements of blended Test Sample 12-1 versus Control Sample 4-1 are given in TABLE 27 (Odor Scores for Test Sample 12-1 versus Control Sample 4-1).

TABLE 25

| | | | | | | | Solution | Solids | CD | EO Add- | Glycerol | |
|---|---|---|---|---|---|---|---|---|---|---|---|---|
| | | | | | | | Add-on | Add-On | Add-On | On | Add-On (kg | Fluff |
| | | Solution | | | | | (g | (g | (kg CD/ | (kg Citral/ | glycerol/ | Moisture |
| | | | Liquid | Soln Wt. | T | | soln/g | solids/g | ADMT | ADMT | ADMT | Increase |
| Sample | Description | Component | Conc | Fraction | (° C.) | pH | fluff) | fluff) | fluff) | fluff) | fluff) | (%) |
| Citral 1.6 | | glycerol | 0.00 | 0.000 | 86C/ | 12.5 | 0.0124 | 0.0022 | 1.8 | 0.400 | 0.0 | 1.0% |
| 0.40 | | water | 1.00 | 0.820 | 86C | | | | | | | |
| 86C/86C | | Subtotal | | 0.820 | | | | | | | | |
| | | β-Cyclodextrin | | 0.148 | | | | | | | | |
| | | Citra | | 0.032 | | | | | | | | |
| | | | | 0.000 | | | | | | | | |
| | | Total | | 1.00 | | | | | | | | |

Mole Ratio (mol EO/mol CD) = 1.625

| | Solution Preparation: | | |
|---|---|---|---|
| MW (g/mol) | | g used | % |

| 1135 | β-Cyclodextrin | 9.0 | 14.8% | 18.0% | Total (g) 11.0 |
|---|---|---|---|---|---|
| 152.2 | Citral | 1.96 | 3.2% | | |
| | | 0.00 | 0.0% | | |
| | Water | 50.0 | 82.0% | | |
| | Total | 61.0 | 100% | | |

TABLE 26

| | | | | | | | Solution | Solids | CD | EO Add- | Glycerol | |
|---|---|---|---|---|---|---|---|---|---|---|---|---|
| | | | | | | | Add-on | Add-On | Add-On | On | Add-On (kg | Fluff |
| | | Solution | | | | | (g | (g | (kg CD/ | (kg EO/ | glycerol/ | Moisture |
| | | | Liquid | Soln Wt. | T | | soln/g | solids/g | ADMT | ADMT | ADMT | Increase |
| Sample | Description | Component | Conc | Fraction | (° C.) | pH | fluff) | fluff) | fluff) | fluff) | fluff) | (%) |
| Citral- | | glycerol | 0.00 | 0.000 | 86C/ | 12.5 | 0.0131 | 0.0024 | 1.9 | 0.46 | 0.0 | 1.1% |
| Coconut | | water | 1.00 | 0.818 | 86C | | | | | | | |
| Fragrance | | Subtotal | | 0.818 | | | | | | | | |
| Blend | | β-Cyclodextrin | | 0.147 | | | | | | | | |
| | | Citral | | 0.016 | | | | | | | | |
| | | Coconut | | 0.019 | | | | | | | | |
| | | Total: | | 1.00 | | | | | | | | |

Mole Ratio (mol EO/mol CD) = 1.625 × 0.5 = 0.8125

| | Solution Preparation: | | |
|---|---|---|---|
| MW (g/mol) | | g used | % |

| | | | | | |
|---|---|---|---|---|---|
| 1135 | β-Cyclodextrin | 9.0 | 14.7% | 18.2% | Total (g) 11.1 |
| 152.2 | Citral | 0.98 | 1.6% | | |
| 180.2 | Coconut | 1.16 | 1.9% | | |
| | Water | 50.0 | 81.8% | | |
| | Total: | 61.1 | 100% | | |

TABLE 27

| | | | | Liquid Dosing Level | | | Odor | |
|---|---|---|---|---|---|---|---|---|
| Triangular Forced-Choice Odor Evaluation | | | time | (g liq/g fluff) | | | Score | |
| Test | 2 Urine Controls | 1 Urine Unknown | (min) | 2.5 | 5 | 10 | Summary | |
| urine/urine | 4-1/urine | 12-1/urine | 10 | | 6± | 6+ | +12 | +24 |
| | 0.40 Adv Biotech Citral | 0.46 equimolar Citral- | 100 | | 6+ | 6+ | +12 | |
| | 1.625 mole ratio | Coconut fragrance blend | | | | | | |
| | pH 12.5 86 C./86 C. | 1.625 mole ratio | | | | | | |
| | | pH 12.5 86 C./86 C. | | | | | | |

### Example 11: Decrease in Solution Mixing Temperature from 86° C. to 50° C. Achieved by Increasing EO-to-CD Mole Ratio from 1.6 to 4.0

Sample 4-1, described above in Example 10, was used as the Control sample in a Triangular Forced-Choice Odor Evaluation versus a new test sample, identified as 52-1, prepared at a lower mixing temperature and a higher mole ratio. A reduction in mixing temperature reduced the solubility and dispersibility of the citral/β-cyclodextrin complex. This reduced dispersibility led to a settling of the precipitated complex at a mixing temperature of 50° C. It was difficult to apply such a dispersion to a substrate, and the odor-control performance was poor. Removing β-cyclodextrin from the solution, with a resulting increase in the citral to β-cyclodextrin mole ratio to a value of 4.0, allowed for an effective dispersion of the solids at 50° C. At 50° C., the dispersion used to make Test Sample 52-1 was milky with no noticeable precipitation or particle settling.

Test Sample 52-1 was prepared using 100% water and a pH adjustment of 12.5 using β-cyclodextrin from Wacker Chemical Co. and citral from Advanced Biotech. The solution was prepared at a citral to β-cyclodextrin mole ratio of 4.0, and mixed and applied at a temperature of 50° C. (indicated as 50 C/50 C in the table). The amount of citral add-on was increased to 0.5 kg of citral per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the substrate was 1.3 wt. %. Because the moisture increase was not much higher than 1 wt. %, no drying step was required.

The odor score of Test Sample 52-1 versus Control Sample 4-1 was +20, out of a possible range of −24 to +24, as determined by the blind, triangular forced-choice test discussed above. The increase in the citral to β-cyclodextrin mole ratio from a value of 1.6 to 4.0 improved odor control performance. It was unexpected to find relatively good dispersibility of the solution at a mixing temperature of only 50° C. It was also unexpected that there was no citral scent on dry sample 52-1 at the high citral to cyclodextrin mole ratio of 4.0. This indicated that most of the volatile citral oil was bound to the substrate under dry storage conditions. Specifications for control Sample 4-1 and Test Sample 52-1 are given in TABLE 25 and TABLE 28 (Test Sample 52-1). Odor score measurements of Test Sample 52-1 versus Control Sample 4-1 are given in TABLE 29.

TABLE 28

| | | | | | | | | | | EO | Glycerol | Fluff |
|---|---|---|---|---|---|---|---|---|---|---|---|---|
| | | | | | | | | | CD | Add-On | Add-On | Mois- |
| | | | | | | | Solution | Solids | Add-On | (kg | (kg | ture |
| | | Solution | | | | | Add-on | Add-On | (kg CD/ | Citral/ | glycerol/ | In- |
| | Descrip- | | Liquid | Soln Wt. | | | (g soln/g | (g solids/ | ADMT | ADMT | ADMT | crease |
| Sample | tion | Component | Conc | Fraction | T (° C.) | pH | fluff) | g fluff) | fluff) | fluff) | fluff) | (%) |
| Citral 12.5 | | glycerol | 0.00 | 0.000 | 50 C./50 C. | 12.5 | 0.0146 | 0.0015 | 0.9 | 0.510 | 0.0 | 1.3% |
| 4.0 0.51 | | water | 1.00 | 0.900 | | | | | | | | |
| 50 C · 50 C. | | Subtotal | | 0.900 | | | | | | | | |
| | | β-Cyclodextrin | | 0.065 | | | | | | | | |
| | | Citral | | 0.035 | | | | | | | | |
| | | | | 0.000 | | | | | | | | |
| | | Total | | 1.00 | | | | | | | | |
| | | Mole Ratio (mol EO/mol CD) = | | | 4.013 | | | | | | | |
| | MW | Solution Preparation: | | | | | | | | | | |
| | (g/mol) | | | | | | | | | | | |
| | | | g used | % | | | | | | | | |
| | 1135 | β-Cyclodextrin | 3.60 | 6.5% | 10.0% | | Total (g) | 5.5 | | | | |
| | 152.2 | Citral | 1.94 | 3.5% | | | | | | | | |
| | | | 0.00 | 0.0% | | | | | | | | |
| | | Glycerin | 0.0 | 0.0% | | | | | | | | |
| | | Water | 50.0 | 90.0% | | | | | | | | |
| | | Total | 55.5 | 100% | | | | | | | | |

TABLE 29

| | | | | Liquid Dosing Level | | | Odor | |
|---|---|---|---|---|---|---|---|---|
| Triangular Forced-Choice Odor Evaluation | | | time | (g liq/g fluff) | | | Score | |
| Test | 2 Urine Controls | 1 Urine Unknown | (min) | 2.5 | 5 | 10 | Summary | |
| urine/urine | 4-1/urine | 52-1/urine | 10 | | 6+ | 2+ | +8 | +20 |
| | 0.40 Adv Biotech Citral | 0.51 Adv Biotech Citral | 100 | | 6+ | 6+ | +12 | |
| | 1.625 equimolar | 4.031 equimolar | | | | | | |
| | pH 12.5 | pH 12.5 | | | | | | |
| | 86 C./86 C. | 50 C./50 C. | | | | | | |

### Example 12: Decrease in Solution pH from 12.5 to 10.9 Achieved at 50° C. for D-Limonene with the Addition of Corn Syrup

A reduction in pH reduced the solubility and dispersibility of the citral/β-cyclodextrin complex, much like the effect of reduced temperature on the solubility and dispersibility. It was possible to improve the dispersibility of the complex at pH 10.9 by adding water to decrease the solids concentration of the solution. This allowed for a reduction in solution pH at a temperature of 50° C., and maintained some odor-control performance, but this approach led to an undesirable increase in moisture content of the fluff pulp, sometimes greater than 2%. An example of this approach is shown as Sample 46-B in TABLE 40. Attempting to find an additive capable of interfering with the crystallization and/or precipitation of the dispersed complex at reduced temperature and pH, it was discovered that small additions of corn syrup (i.e., glucose) were effective at maintaining a sufficiently stable, milky dispersion at pH 10.9 and 50° C. with no noticeable precipitation or particle settling. It is believed that the smaller glucose molecules interacted with the outer surface of the chemically-similar, β-cyclodextrin complex to reduce the rate and amount of crystallization and/or precipitation of the complex. The favorable effect of the addition of glucose to the solution was demonstrated using a d-limonene and β-cyclodextrin complex in the following example.

Test Sample 65-1 was prepared using 82.7% water and 9.2% Karo Light corn syrup at 50° C. and pH 10.9 using β-cyclodextrin from Wacker Chemical Co. and d-limonene, in place of citral, from Florida Chemical Company. The water content of the corn syrup was estimated at 25 wt. % for calculations in TABLE 28. The solution was prepared at a d-limonene to β-cyclodextrin mole ratio of 4.0, and mixed and applied at a temperature of 50° C. Corn syrup was added to the water before adjusting pH. Any fine precipitate formed remained suspended for hours. It was a very easy solution/suspension to handle and spray during the treatment of the pulp sheet. The amount of d-limonene add-on was 0.5 kg of d-limonene per air-dried metric ton (“ADMT”) of fluff. Fluff pulp in sheet form was an untreated southern pine Kraft pulp sold under the trade name RAYFLOC® JLDE that was obtained from Rayonier Advanced Materials Jesup, GA. The calculated increase in moisture content of the fluff pulp substrate was 1.6 wt. %. No drying step was required.

Test Sample 65-1 was tested for odor versus Control Sample 52-1. Sample 52-1 was used as a Test Sample in Example 11. In paired comparisons, Sample 52-1 performed better in odor control than Sample 4-1 in Example 11 and Sample 65-1 performed better than Sample 52-1 in Example 12. The odor score of Test Sample 65-1 versus Control Sample 52-1 was +24, out of a possible range of −24 to +24, as determined by the blind, triangular forced-choice test method discussed above. The addition of 9.2 wt. % of corn syrup to the solution with the use of d-limonene greatly improved the dispersibility of the complex in the solution and provided a very good masking of urine malodor. There was no scent on dry samples of 65-1. Specifications for Control Sample 52-1 and Test Sample 65-1 are given in TABLE 28 (above) and TABLE 30 (Test Sample 65-1). Odor score measurements of Test Sample 65-1 versus Control Sample 52-1 are given in TABLE 31 (Odor Scores for Test Sample 65-1 versus Control Sample 52-1).

TABLE 30

| | | | | | | | | | | | Corn | Fluff |
|---|---|---|---|---|---|---|---|---|---|---|---|---|
| | | | | | | | | | CD | EO | Syrup | Mois- |
| | | | | | | | Solution | Solids | Add-On | Add-On | Add-On | ture |
| | | Solution | | | | | Add-on | Add-On | (kg CD/ | (kg EO/ | (kg syrup/ | In- |
| | Descrip- | | Liquid | Soln Wt. | | | (g soln/g | (g solids/ | ADMT | ADMT | ADMT | crease |
| Sample | tion | Component | Conc | Fraction | T (° C.) | pH | fluff) | g fluff) | fluff) | fluff) | fluff) | (%) |
| Limonene | | glucose | 0.08 | 0.069 | 50 C./50 C. | 10.9 | 0.0192 | 0.0016 | 1.06 | 0.51 | 1.3 | 1.6% |
| 10.9 | | water | 0.93 | 0.849 | | | | | | | | |
| 4.0 0.51 | | Subtotal: | | 0.918 | | | | | | | | |
| 50 C. · 50 C. | | β-Cyclodextrin | | 0.055 | | | | | | | | |
| | | limonene | | 0.027 | | | | | | | | |
| | | | | 0.000 | | | | | | | | |
| | | Total | | 1.00 | | | | | | | | |
| | | Mole Ratio (mol EO/mol CD) = | | | 4.013 | | | | | | | |
| | MW | Solution Preparation: | | | | | | | | | | |
| | (g/mol) | | | | | | | | | | | |
| | | | g used | % | | | | | | | | |
| | 1135 | β-Cyclodextrin | 3.60 | 5.5% | 8.2% | | Total (g) | 5.3 | | | | |
| | 136.2 | limonene | 1.73 | 2.7% | | | | | | | | |
| | | | 0.00 | 0.0% | | | | | | | | |
| | | Corn syrup | 4.5 | 6.9% | 9.2% | | | | | | | |
| | | glucose | | | | | | | | | | |
| | | Corn syrup | 1.5 | 2.3% | | | | | | | | |
| | | water | | | | | | | | | | |
| | | Water | 54.0 | 82.7% | | | | | | | | |
| | | Total | 65.3 | 100% | | | | | | | | |
| | | Corn syrup | 6.0 | | | | | | | | | |
| | | total | | | | | | | | | | |

TABLE 31

| | | | | Liquid Dosing Level | | | Odor | |
|---|---|---|---|---|---|---|---|---|
| Triangular Forced-Choice Odor Evaluation | | | time | (g liq/g fluff) | | | Score | |
| Test | 2 Urine Controls | 1 Urine Unknown | (min) | 2.5 | 5 | 10 | Summary | |
| urine/urine | 52-1/urine | 65-1/urine | 10 | | 6± | 6+ | +12 | +24 |
| | 0.51 Adv Biotech Citral | 0.51 d-limonene | 100 | | 6+ | 6+ | +12 | |
| | pH 10.9 | pH 10.9 | | | | | | |
| | 4.013 mole ratio | 4.013 mole ratio | | | | | | |
| | 50 C./50 C. | 50 C./50 C. | | | | | | |

### Example 13: Decrease in Solution pH from 12.5 to 10.9 Achieved at 50° C. for an Equimolar D-Limonene/Octanol Blend with the Addition of Corn Syrup

A sample similar to the one described in Example 12 was prepared using an equimolar blend of d-limonene and octanol in place of 100% d-limonene. The water content of the corn syrup was estimated at 25 wt. % for calculations in TABLE 30. Test Sample 67-1 was tested for odor versus Control Sample 52-1. Sample 52-1 was used as a Test Sample in Example 11 and a Control Sample in Example 12. The odor score of Test Sample 67-1 versus Control Sample 52-1 was +24, out of a possible range of −24 to +24, as determined by the blind, triangular forced-choice test method discussed above. The addition of 9.2 wt. % of corn syrup to the solution and the use of an equimolar blend of d-limonene and octanol greatly improved the dispersibility of the complex in the solution and provided very good masking of urine malodor. There was no scent on dry samples of 67-1. Specifications for Control Sample 52-1 and Test Sample 67-1 are given in TABLE 28 and TABLE 32 (Test Sample 67-1). Odor score measurements of Test Sample 67-1 versus Control Sample 52-1 are given in TABLE 33 (Odor Scores for Test Sample 67-1 versus Control Sample 52-1).

TABLE 32

| | | | | | | | | | | | Glucose | Fluff |
|---|---|---|---|---|---|---|---|---|---|---|---|---|
| | | | | | | | | | CD | EO | Add-On | Mois- |
| | | | | | | | Solution | Solids | Add-On | Add-On | (kg | ture |
| | | Solution | | | | | Add-on | Add-On | (kg CD/ | (kg EO/ | syrup/ | In- |
| | Descrip- | | Liquid | Soln Wt. | | | (g soln/g | (g solids/ | ADMT | ADMT | ADMT | crease |
| Sample | tion | Component | Conc | Fraction | T (° C.) | pH | fluff) | g fluff) | fluff) | fluff) | fluff) | (%) |
| Limonene- | | glucose | 0.08 | 0.069 | 50 C./50 C. | 10.9 | 0.0196 | 0.0016 | 1.08 | 0.51 | 1.4 | 1.7% |
| Octanol | | water | 0.93 | 0.850 | | | | | | | | |
| Blend 10.9 | | Subtotal | | 0.919 | | | | | | | | |
| 4.0 0.51 | | β-Cyclodextrin | | 0.055 | | | | | | | | |
| 50 C. · 50 C. w | | limonene | | 0.013 | | | | | | | | |
| 54 g water and | | octanol | | 0.013 | | | | | | | | |
| 6 g syrup | | Total | | 1.00 | | | | | | | | |
| | | Mole Ratio (mol EO/mol CD) = | | | 4.013 | | Mole Ratio (mol limonene/mol octanol) = | | | | 1 | |
| | MW | Solution Preparation: | | | | | | | | | | |
| | (g/mol) | | | | | | | | | | | |
| | | | g used | % | | | | | | | | |
| | 1135 | β-Cyclodextrin | 3.60 | 5.5% | 8.1% | | Total (g) | 5.3 | | | | |
| | 136.2 | limonene | 0.87 | 1.3% | | | | | | | | |
| | 130.2 | octanol | 0.83 | 1.3% | | | | | | | | |
| | | Corn syrup | 4.5 | 6.9% | 9.2% | | | | | | | |
| | | glucose | | | | | | | | | | |
| | | Corn syrup | 1.5 | 2.3% | | | | | | | | |
| | | water | | | | | | | | | | |
| | | Water | 54.0 | 82.7% | | | | | | | | |
| | | Total | 65.3 | 100% | | | | | | | | |
| | | Corn syrup | 6.0 | | | | | | | | | |
| | | total | | | | | | | | | | |

TABLE 33

| | | | | Liquid Dosing Level | | | Odor | |
|---|---|---|---|---|---|---|---|---|
| Triangular Forced-Choice Odor Evaluation | | | time | (g liq/g fluff) | | | Score | |
| Test | 2 Urine Controls | 1 Urine Unknown | (min) | 2.5 | 5 | 10 | Summary | |
| urine/urine | 52-1/urine | 67-1/urine | 10 | | 6+ | 6+ | +12 | +24 |
| | 0.51 Adv Biotech Citral | 0.51 d-limonene/octanol | 100 | | 6+ | 6+ | +12 | |
| | pH 12.5 | pH 10.9 | | | | | | |
| | 4.013 mole ratio | 4.013 mole ratio | | | | | | |
| | 50 C./50 C. | 50 C./50 C. | | | | | | |

### Example 14: Further Increase in EO/CD Mole Ratio and Decreases in pH and Mixing Temperature

Samples similar to that of Sample 67-1 described in Example 13 (TABLE 32) were made to extend the range of the key variables of pH, mixing temperature and EO/CD mole ratio. These samples were prepared using equimolar blends of d-limonene and octanol. The solutions used to make these samples contained 9.2% to 9.4% of corn syrup, with the water content of the corn syrup estimated at 25 wt. %. The solution pH and mixing temperature of Sample 67-1 was pH 10.9 and 50 C, respectively. The pH of the solution used to make Sample 68-1 was pH 7.0 and the mixing temperature of the solution used to make Sample 69-1 was 20 C. Both of these samples had a solution concentration of CD and EO of 8.1%, like that of Sample 67-1. The solution used to make Sample 74-1 was made at an increased EO/CD mole ratio of 6.0, versus the EO/CD mole ratio of 4.0 for 67-1, and the pH and mixing temperature for 74-1 was 10.9 and 50 C, respectively. Sample 74-1 had a solution concentration of CD and EO of only 6.4% because of the reduction in the amount of cyclodextrin. The specification of Sample 74-1 is given in TABLE 34.

TABLE 34

| | | | | | | | | | | | Glucose | Fluff |
|---|---|---|---|---|---|---|---|---|---|---|---|---|
| | | | | | | | | | CD | EO | Add-On | Mois- |
| | | | | | | | Solution | Solids | Add-On | Add-On | (kg | ture |
| | | Solution | | | | | Add-on | Add-On | (kg CD/ | (kg EO/ | syrup/ | In- |
| | Descrip- | | Liquid | Soln Wt. | | | (g soln/g | (g solids/ | ADMT | ADMT | ADMT | crease |
| Sample | tion | Component | Conc | Fraction | T (° C.) | pH | fluff) | g fluff) | fluff) | fluff) | fluff) | (%) |
| Limonene- | | glucose | 0.08 | 0.070 | 50 C./50 C. | 10.9 | 0.0193 | 0.0012 | 0.72 | 0.51 | 1.4 | 1.7% |
| Octanol | | water | 0.93 | 0.866 | | | | | | | | |
| Blend 10.9 | | Subtotal | | 0.936 | | | | | | | | |
| 6.0 0.51 | | β-Cyclodextrin | | 0.038 | | | | | | | | |
| 50 C. · 50 C. w | | limonene | | 0.014 | | | | | | | | |
| 54 g water and | | octanol | | 0.013 | | | | | | | | |
| 6 g syrup | | Total | | 1.00 | | | | | | | | |
| | | Mole Ratio (mol EO/mol CD) = | | | 6.000 | | Mole Ratio (mol limonene/mol octanol) = | | | | 1 | |
| | MW | Solution Preparation: | | | | | | | | | | |
| | (g/mol) | | | | | | | | | | | |
| | | | g used | % | | | Total (g) | 4.1 | | | | |
| | 1135 | β-Cyclodextrin | 2.41 | 3.8% | 6.4% | | | | | | | |
| | 136.2 | limonene | 0.87 | 1.4% | | | | | | | | |
| | 130.2 | octanol | 0.83 | 1.3% | | | | | | | | |
| | | Corn syrup | 4.5 | 7.0% | 9.4% | | | | | | | |
| | | glucose | | | | | | | | | | |
| | | Corn syrup | 1.5 | 2.3% | | | | | | | | |
| | | water | | | | | | | | | | |
| | | Water | 54.0 | 84.2% | | | | | | | | |
| | | Total | 64.1 | 100% | | | | | | | | |
| | | Corn syrup | 6.0 | | | | | | | | | |
| | | total | | | | | | | | | | |

Test Samples 68-1, 69-1, and 74-1 were tested for urine malodor versus Control Sample 67-1. The odor scores of Test Samples 68-1, 69-1, and 74-1 versus Control Sample 67-1, as determined by the blind, triangular forced-choice test method, are summarized in TABLE 35.

TABLE 35

| | | | | | | | Solution Concentrations | | | | |
|---|---|---|---|---|---|---|---|---|---|---|---|
| | Key Factors | | | | | | | Soln | | | |
| | | Mixing/ | | | Conc | | Soln | Conc of | Soln | | |
| | EO Add- | Appn | EO/CD | | Corn | | Conc of | Corn | Conc of | Moisture | |
| | on (kg/ | Temp | Mole | | Syrup | g water/ | CD & EO | Syrup | Water | Increase | Odor-control |
| Sample | ADMT) | (C.) | Ratio | pH | (wt %) | g syrup | (wt %) | (wt %) | (wt %) | (wt %) | Score vs. 67-1 |
| 67-1 | 0.51 | 50 C. | 4.0 | 10.9 | 9.2 | 54/6 | 8.1 | 9.2 | 85.0 | 1.7% | — |
| 68-1 | 0.51 | 50 C. | 4.0 | 7.0 | 9.2 | 54/6 | 8.1 | 9.2 | 85.0 | 1.7% | −20 |
| 69-1 | 0.51 | 20 C. | 4.0 | 10.9 | 9.2 | 54/6 | 8.1 | 9.2 | 85.0 | 1.7% | −6 |
| 74-1 | 0.51 | 50 C. | 6.0 | 10.9 | 9.4 | 54/6 | 6.4 | 9.4 | 86.6 | 1.7% | −12 |

Odor scores for Test Samples 68-1, 69-1, and 74-1 showed that none of these samples were as good as Control Sample 67-1, but their odor-control performance was still relatively good. The odor-control performance of Sample 69-1 made with a solution mixing temperature of 20 C was almost as good as the odor-control performance of Control Sample 67-1. The reduction of pH and the increase in mole ratio had a greater negative effect on odor-control performance than that of temperature. These samples were used to establish ranges of the key factors for the optimal odor-control performance of samples containing corn syrup.

### Example 15: Further Decrease in Solution Concentration of Corn Syrup

Samples similar to that of Sample 67-1 described in Example 13 (TABLE 32) were made to extend the range of the key variable of solution concentration of corn syrup. These samples were prepared using equimolar blends of d-limonene and octanol. The solutions used to make these samples contained 9.2% to 3.3% of corn syrup, with the water content of the corn syrup estimated at 25 wt. %. The pH of the solution used to make the samples was pH 10.9, the mixing temperature was 50 C and the EO/CD mole ratio was 4.0. Solution concentrations of CD and EO ranged from 8.1% to 13.8%. Specifications of Samples 72-1, 70-1, and 82-1 are given in TABLE 36, 37, and 38.

TABLE 36

| | | | | | | | | | | | Glucose | Fluff |
|---|---|---|---|---|---|---|---|---|---|---|---|---|
| | | | | | | | | | CD | EO | Add-On | Mois- |
| | | | | | | | Solution | Solids | Add-On | Add-On | (kg | ture |
| | | Solution | | | | | Add-on | Add-On | (kg CD/ | (kg EO/ | syrup/ | In- |
| | Descrip- | | Liquid | Soln Wt. | | | (g soln/g | (g solids/ | ADMT | ADMT | ADMT | crease |
| Sample | tion | Component | Conc | Fraction | T (° C.) | pH | fluff) | g fluff) | fluff) | fluff) | fluff) | (%) |
| Limonene- | | glucose | 0.07 | 0.062 | 50 C./50 C. | 10.9 | 0.0146 | 0.0016 | 1.08 | 0.51 | 0.9 | 1.2% |
| Octanol | | water | 0.93 | 0.829 | | | | | | | | |
| Blend 10.9 | | Subtotal | | 0.891 | | | | | | | | |
| 4.0 0.51 | | β-Cyclodextrin | | 0.074 | | | | | | | | |
| 50 C. · 50 C. w | | limonene | | 0.018 | | | | | | | | |
| 39 g water and | | octanol | | 0.017 | | | | | | | | |
| 4 g syrup | | Total | | 1.00 | | | | | | | | |
| | | Mole Ratio (mol EO/mol CD) = | | | 4.014 | | Mole Ratio (mol limonene/mol octanol) = | | | | 1 | |
| | MW | Solution Preparation: | | | | | | | | | | |
| | (g/mol) | | | | | | | | | | | |
| | | | g used | % | | | | | | | | |
| | 1135 | β-Cyclodextrin | 3.60 | 7.4% | 10.9% | | Total (g) | 5.3 | | | | |
| | 136.2 | limonene | 0.87 | 1.8% | | | | | | | | |
| | 130.2 | octanol | 0.83 | 1.7% | | | | | | | | |
| | | Corn syrup | 3.0 | 6.2% | 8.3% | | | | | | | |
| | | glucose | | | | | | | | | | |
| | | Corn syrup | 1.0 | 2.1% | | | | | | | | |
| | | water | | | | | | | | | | |
| | | Water | 39.1 | 80.8% | | | | | | | | |
| | | Total | 48.4 | 100% | | | | | | | | |
| | | Corn syrup | 4.0 | | | | | | | | | |
| | | total | | | | | | | | | | |

TABLE 37

| | | | | | | | | | | | Glucose | Fluff |
|---|---|---|---|---|---|---|---|---|---|---|---|---|
| | | | | | | | | | CD | EO | Add-On | Mois- |
| | | | | | | | Solution | Solids | Add-On | Add-On | (kg | ture |
| | | Solution | | | | | Add-on | Add-On | (kg CD/ | (kg EO/ | syrup/ | In- |
| | Descrip- | | Liquid | Soln Wt. | | | (g soln/g | (g solids/ | ADMT | ADMT | ADMT | crease |
| Sample | tion | Component | Conc | Fraction | T (° C.) | pH | fluff) | g fluff) | fluff) | fluff) | fluff) | (%) |
| Limonene- | | glucose | 0.05 | 0.039 | 50 C./50 C. | 10.9 | 0.0115 | 0.0016 | 1.08 | 0.51 | 0.5 | 0.9% |
| Octanol | | water | 0.95 | 0.823 | | | | | | | | |
| Blend 10.9 | | Subtotal | | 0.862 | | | | | | | | |
| 4.0 0.51 | | β-Cyclodextrin | | 0.094 | | | | | | | | |
| 50 C. · 50 C. w | | limonene | | 0.023 | | | | | | | | |
| 31.0 g water | | octanol | | 0.022 | | | | | | | | |
| and 2 g | | Total | | 1.00 | | | | | | | | |
| | | Mole Ratio (mol EO/mol CD) = | | | 4.014 | | Mole Ratio (mol limonene/mol octanol) = | | | | 1 | |
| | MW | Solution Preparation: | | | | | | | | | | |
| | (g/mol) | | | | | | | | | | | |
| | | | g used | % | | | | | | | | |
| | 1135 | β-Cyclodextrin | 3.60 | 9.4% | 13.8% | | Total (g) | 5.3 | | | | |
| | 136.2 | limonene | 0.87 | 2.3% | | | | | | | | |
| | 130.2 | octanol | 0.83 | 2.2% | | | | | | | | |
| | | Corn syrup | 1.5 | 3.9% | 5.2% | | | | | | | |
| | | glucose | | | | | | | | | | |
| | | Corn syrup | 0.5 | 1.3% | | | | | | | | |
| | | water | | | | | | | | | | |
| | | Water | 31.0 | 80.9% | | | | | | | | |
| | | Total | 38.3 | 100% | | | | | | | | |
| | | Corn syrup | 2.0 | | | | | | | | | |
| | | total | | | | | | | | | | |

TABLE 38

| | | | | | | | | | | | Glucose | Fluff |
|---|---|---|---|---|---|---|---|---|---|---|---|---|
| | | | | | | | | | CD | EO | Add-On | Mois- |
| | | | | | | | Solution | Solids | Add-On | Add-On | (kg | ture |
| | | Solution | | | | | Add-on | Add-On | (kg CD/ | (kg EO/ | syrup/ | In- |
| | Descrip- | | Liquid | Soln Wt. | | | (g soln/g | (g solids/ | ADMT | ADMT | ADMT | crease |
| Sample | tion | Component | Conc | Fraction | T (° C.) | pH | fluff) | g fluff) | fluff) | fluff) | fluff) | (%) |
| Limonene- | | glucose | 0.03 | 0.024 | 50 C./50 C. | 10.9 | 0.0184 | 0.0016 | 1.08 | 0.51 | 0.5 | 1.6% |
| Octanol | | water | 0.97 | 0.889 | | | | | | | | |
| Blend 10.9 | | Subtotal | | 0.914 | | | | | | | | |
| 4.0 0.51 | | β-Cyclodextrin | | 0.059 | | | | | | | | |
| 50 C. · 50 C. w | | limonene | | 0.014 | | | | | | | | |
| 54 g water and | | octanol | | 0.014 | | | | | | | | |
| 2 g syrup | | Total | | 1.00 | | | | | | | | |
| | | Mole Ratio (mol EO/mol CD) = | | | 4.014 | | Mole Ratio (mol limonene/mol octanol) = | | | | 1 | |
| | MW | Solution Preparation: | | | | | | | | | | |
| | (g/mol) | | | | | | | | | | | |
| | | | g used | % | | | | | | | | |
| | 1135 | β-Cyclodextrin | 3.60 | 5.9% | 8.6% | | Total (g) | 5.3 | | | | |
| | 136.2 | limonene | 0.87 | 1.4% | | | | | | | | |
| | 130.2 | octanol | 0.83 | 1.4% | | | | | | | | |
| | | Corn syrup | 1.5 | 2.4% | 3.3% | | | | | | | |
| | | glucose | | | | | | | | | | |
| | | Corn syrup | 0.5 | 0.8% | | | | | | | | |
| | | water | | | | | | | | | | |
| | | Water | 54.0 | 88.1% | | | | | | | | |
| | | Total | 61.3 | 100% | | | | | | | | |
| | | Corn syrup | 2.0 | | | | | | | | | |
| | | total | | | | | | | | | | |

Test Samples 72-1, 70-1, and 82-1 were tested for urine malodor versus Control Sample 67-1. The odor scores of the Test Samples versus Control Sample 67-1, as determined by the blind, triangular forced-choice test method, are summarized in TABLE 39.

TABLE 39

| | | | | | | | Solution Concentrations | | | | |
|---|---|---|---|---|---|---|---|---|---|---|---|
| | Key Factors | | | | | | | Soln | | | |
| | | Mixing/ | | | Conc | | Soln | Conc of | Soln | | |
| | EO Add- | Appn | EO/CD | | Corn | | Conc of | Corn | Conc of | Moisture | |
| | on (kg/ | Temp | Mole | | Syrup | g water/ | CD & EO | Syrup | Water | Increase | Odor-control |
| Sample | ADMT) | (C.) | Ratio | pH | (wt %) | g syrup | (wt %) | (wt %) | (wt %) | (wt %) | Score vs. 67-1 |
| 67-1 | 0.51 | 50 C. | 4.0 | 10.9 | 9.2 | 54/6 | 8.1 | 9.2 | 85.0 | 1.7% | — |
| 72-1 | 0.51 | 50 C. | 4.0 | 10.9 | 8.3 | 39/4 | 10.9 | 8.3 | 82.9 | 1.2% | −12 |
| 70-1 | 0.51 | 50 C. | 4.0 | 10.9 | 5.2 | 31/2 | 13.8 | 5.2 | 82.3 | 0.9% | −24 |
| 82-1 | 0.51 | 50 C. | 4.0 | 10.9 | 3.3 | 54/2 | 8.6 | 3.3 | 88.9 | 1.6% | −8 |

Odor-control performance of Samples 67-1, 72-1 and 70-1 decreased with increasing solution concentration of CD and EO, and decreasing solution concentration of corn syrup. Sample 82-1 illustrated the point that a low concentration of corn syrup in the range of 3.3% was effective when the total solution concentration of CD and EO was low at about 8% to 9%.

### Example 16: Summary of Samples in Examples 10 to 17

The relative odor-control performance of the samples in Examples 10 to 17 was also summarized using an odor performance score that provided a continuous scale of performance based on the multiple paired comparisons. For example, if Sample 4-1 was +24 compared to conventional fluff, and Sample 52-1 was +20 compared to Sample 4-1, and Sample 67-1 was +24 compared to Sample 52-1, then Sample 67-1 was +68 compared to conventional fluff. The scale is not linear, with a given quantitative difference at the low end of the scale being more meaningful than the same difference at the upper end. These Continuous Odor-Control Scores of the samples are shown in TABLE 40. Ranges of the key factors for optimal odor-control performance are also shown in the table. The key factors are total essential oil (EO) add-on to substrate, solution mixing temperature, essential oil to cyclodextrin mole ratio in solution, solution pH, and corn syrup concentration in solution.

TABLE 40

| | | | | | | | | Solution Concentrations | | | | | |
|---|---|---|---|---|---|---|---|---|---|---|---|---|---|
| | KEY FACTORS | | | | | | | | Soln | | | Mois- | Contin- |
| | | EO | Mixing/ | | | Conc | | Soln | Conc of | Soln | Soln | ture | uous |
| | | Add-on | Appn | EO/CD | | Corn | | Conc of | Corn | Conc of | Conc of | In- | Odor- |
| | Glycerol/ | (kg/ | Temp | Mole | | Syrup | g water/ | CD & EO | Syrup | Glucose | Water | crease | Control |
| Sample | water | ADMT) | (C.) | Ratio | pH | (wt %) | g syrup | (wt %) | (wt %) | (wt %) | (wt %) | (wt %) | Score |
| Preferred | 0/100 | 0.2 | 20 C. | 1.5 | 9.0 | 3.3 | | | | | | | |
| Ranges | | to | to | to | to | to | | | | | | | |
| | | 2.0 | 86 C. | 6.0 | 12.5 | 9.4 | | | | | | | |
| Conventional fluff | | | | | | | | | | | | | 0 |
| Ex 7 | 85/15 | 2.0 | 86 C. | 1.5 | 12.5 | 0 | 50/0 | 21.6 | 0 | 0 | 11.8 | 0.7% | 24 |
| Ex 8 | 85/15 | 0.40 | 86 C. | 1.5 | 12.5 | 0 | 50/0 | 21.6 | 0 | 0 | 11.8 | 0.1% | 24 |
| Ex 9 | 0/100 | 0.40 | 86 C. | 1.5 | 12.5 | 0 | 50/0 | 17.8 | 0 | 0 | 82.2 | 1.1% | 24 |
| Ex 10 (4-1) | 0/100 | 0.40 | 86 C. | 1.6 | 12.5 | 0 | 50/0 | 18.0 | 0 | 0 | 82.0 | 1.0% | 24 |
| Ex 10 (12-1) | 0/100 | 0.46 | 86 C. | 1.6 | 12.5 | 0 | 50/0 | 18.2 | 0 | 0 | 81.8 | 1.1% | 48 |
| Ex 11 (52-1) | 0/100 | 0.51 | 50 C. | 4.0 | 12.5 | 0 | 50/0 | 10.0 | 0 | 0 | 90.0 | 1.3% | 44 |
| (46-B) | 0/100 | 0.51 | 50 C. | 4.0 | 10.9 | 0 | 70/0 | 7.0 | 0 | 0 | 93.0 | 2.1% | 34 |
| Ex 13 (67-1) | 0/100 | 0.51 | 50 C. | 4.0 | 10.9 | 9.2 | 54/6 | 8.1 | 9.2 | 6.9 | 85.0 | 1.7% | 68 |
| Ex 14 (74-1) | 0/100 | 0.51 | 50 C. | 6.0 | 10.9 | 9.4 | 54/6 | 6.4 | 9.4 | 7.0 | 86.6 | 1.7% | 56 |
| Ex 14 (68-1) | 0/100 | 0.51 | 50 C. | 4.0 | 7.0 | 9.2 | 54/6 | 8.1 | 9.2 | 6.9 | 85.0 | 1.7% | 48 |
| Ex 14 (69-1) | 0/100 | 0.51 | 20 C. | 4.0 | 10.9 | 9.2 | 54/6 | 8.1 | 9.2 | 6.9 | 85.0 | 1.7% | 62 |
| Ex 15 (72-1) | 0/100 | 0.51 | 50 C. | 4.0 | 10.9 | 8.3 | 39/4 | 10.9 | 8.3 | 6.2 | 82.9 | 1.2% | 56 |
| Ex 15 (70-1) | 0/100 | 0.51 | 50 C. | 4.0 | 10.9 | 5.2 | 31/2 | 13.8 | 5.2 | 3.9 | 82.3 | 0.9% | 44 |
| Ex 15 (82-1) | 0/100 | 0.51 | 50 C. | 4.0 | 10.9 | 3.3 | 54/2 | 8.6 | 3.3 | 2.4 | 88.9 | 1.6% | 60 |

| | | | | | | | | |
|---|---|---|---|---|---|---|---|---|
| 0/100 | Range for Performance Score 48-68 | min. | 6.4 | 3.3 | 2.4 | 82.3 | 0.9% | 48 |
| only | (Level 0 = conventional fluff; 68 = best) | max. | 18.2 | 9.4 | 7.0 | 88.9 | 1.6% | 68 |
| | Range for Performance Score 24-68 | min. | 6.4 | 0 | 0 | 82.0 | 0.9% | 24 |
| | (Level 0 = conventional fluff; 68 = best) | max. | 18.2 | 9.4 | 7.0 | 93.0 | 2.1% | 68 |

### Example 17: Comparison of Odor-Control Fluff Samples with a Conventional Fluff

All of the test and control samples in Examples 10 to 16 scored a perfect +12 score versus conventional fluff in the odor-control test. This indicated that the samples in Examples 10 to 16 masked urine malodor much better than conventional fluff. TABLE 41 below provides an example of odor scores for two of the test samples versus conventional fluff control samples.

TABLE 41

| | | | | Liquid Dosing Level | | | Odor |
|---|---|---|---|---|---|---|---|
| Triangular Forced-Choice Odor Evaluation | | | time | (g liq/g fluff) | | | Score |
| Test | 2 Controls | 1 Unknown | (min) | 2.5 | 5 | 10 | Summary |
| urine/urine | Rayfloc/urine | Example 10 | 10 | | 6+ | | +12 |
| | | 4-1/urine | 100 | | 6+ | | |
| | | 0.40 Adv Biotech Citral | | | | | |
| | | 1.625 mole ratio | | | | | |
| | | pH 12.5 86 C./86 C. | | | | | |
| urine/urine | Rayfloc/urine | Example 13 | 10 | | 6+ | | +12 |
| | | 67-1/urine | 100 | | 6+ | | |
| | | 0.51 d-limonene/octanol | | | | | |
| | | 4.013 mole ratio | | | | | |
| | | pH 10.9 50 C./50 C. | | | | | |

In the foregoing description, the solutions containing cyclodextrins for imparting odor control to materials used in absorbent products and associated products and methods of the present application have been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification, unless the context requires otherwise, the word “comprise” and its variations, such as “comprises” and “comprising,” will be understood to imply the inclusion of a stated item, element or step or group of items, elements or steps, but not the exclusion of any other item, element or step or group of items, elements or steps. Furthermore, the indefinite article “a” or “an” is meant to indicate one or more of the item, element, or step modified by the article.

1. A substrate comprising an odor-controlling solution applied thereupon,
wherein the odor-controlling solution comprises:
water,
a cyclodextrin,
an odor-controlling compound, and
optionally, a polar organic solvent,
wherein the substrate comprises at least 0.2 kg of the odor-controlling compound per 1000 kg of the substrate, and
wherein the substrate has an increase in moisture content of less than 2 wt. % as a result of the odor-controlling solution having been applied thereupon.

2. The substrate according to claim 1, wherein the odor-controlling solution further comprises a stabilizing additive.

3. The substrate according to claim 1, wherein the odor-controlling solution comprises 1.5 moles to 6 moles of the odor-controlling compound per mole of the cyclodextrin.

4. The substrate according to claim 1, wherein the odor-controlling solution has a pH greater than 9 and less than 12.5.

5. The substrate according to claim 1, wherein the odor-controlling solution comprises about 6 wt. % to about 19 wt. % of the cyclodextrin and the odor-controlling compound.

6. The substrate according to claim 1, wherein the odor-controlling compound is selected from the group consisting of citral, coconut fragrance oil, d-limonene, octanol, and combinations thereof.

7. The substrate according to claim 1, wherein the substrate has an increase in moisture content of less than 1.7 wt % as a result of the odor-controlling solution having been applied thereupon.

8. The substrate according to claim 3,
wherein the odor-controlling solution comprises 3.5 moles to 4.5 moles of the odor-controlling compound per mole of the cyclodextrin, and
wherein, before the odor-controlling solution is applied to the substrate, the odor-controlling solution is mixed at a temperature at or greater than 20° C. such that a dispersion forms that contains a complex of the cyclodextrin and the odor-controlling compound.

9. The substrate according to claim 2, wherein the odor-controlling solution comprises about 0.1 wt. % to about 14 wt. % of the stabilizing additive.

10. The substrate according to claim 2, wherein the stabilizing additive is selected from the group consisting of corn syrup, glucose, high fructose corn syrup, trehalose, sucrose, sorbitol, amino acids, derivatives thereof, and combinations thereof.

11. The substrate according to claim 2,
wherein the stabilizing additive is selected from the group consisting of corn syrup, glucose, high fructose corn syrup, derivatives thereof, and combinations thereof, and
wherein the odor-controlling solution comprises about 2 wt. % to about 10 wt % of the stabilizing additive.

12. The substrate according to claim 2,
wherein the stabilizing additive is trehalose, and
wherein the odor-controlling solution comprises about 0.1 wt. % to about 1.0 wt % of the stabilizing additive.

13. The substrate according to claim 1,
wherein the odor-controlling solution further comprises glucose and/or corn syrup as a stabilizing additive,
wherein the odor-controlling solution comprises about 2.4 wt. % to about 9.4 wt. % of the stabilizing additive,
wherein the odor-controlling solution comprises about 3.5 moles to about 6 moles of the odor-controlling compound per mole of the cyclodextrin,
wherein the odor-controlling solution has a pH greater than 9 and less than 12.5,
wherein the odor-controlling compound is a blend of compounds selected from the group consisting of
a blend of citral and coconut fragrance oil,
a blend of d-limonene and coconut fragrance oil,
a blend of citral and d-limonene, and
a blend of octanol and d-limonene,
wherein, before the odor-controlling solution is applied to the substrate, the odor-controlling solution is mixed at a temperature at or greater than 20° C. such that a dispersion forms that contains a complex of the cyclodextrin and the odor-controlling compound, and
wherein the substrate has an increase in moisture content of less than 1.7 wt. % as a result of the odor-controlling solution having been applied thereupon.

14. An article comprising the substrate according to claim 1.

15. A method of preparing an odor-controlling substrate, comprising:
a) providing a substrate,
b) forming an odor-controlling solution comprising water, a cyclodextrin, an odor-controlling compound, a stabilizing additive, and optionally, a polar organic solvent,
wherein the odor-controlling solution formed in b) is a dispersion that contains a complex of the cyclodextrin and the odor-controlling compound,
c) applying the odor-controlling solution onto the substrate to obtain the odor-controlling substrate,
wherein, after c), the odor-controlling substrate comprises at least 0.2 kg of the odor-controlling compound per 1000 kg of the odor-controlling substrate, and
wherein the substrate has an increase in moisture content of less than 1.7 wt. % as a result of the odor-controlling solution being applied thereupon.

16. A substrate comprising an odor-controlling solution applied thereupon,
wherein the odor-controlling solution comprises:
water,
a cyclodextrin,
an odor-controlling compound, and
a polar organic solvent,
wherein the substrate comprises at least 0.2 kg of the odor-controlling compound per 1000 kg of the substrate.

17. The substrate according to claim 16, wherein the substrate has an increase in moisture content of less than 1 wt. % as a result of the odor-controlling solution having been applied thereupon.

18. The substrate according to claim 16, wherein the substrate is
a fluff pulp in the form of a compressed sheet or an air-laid fiber form,
a synthetic or cellulosic fiber spun-bond or carded nonwoven, woven fabric, or solid material, or
an absorbent article,
wherein, when the substrate is the solid material, the solid material is selected from the group consisting of polymeric foam, leather, and plastic film, and
wherein, the substrate material is the absorbent article, the absorbent article is an adult incontinence control pad, feminine hygiene pad, pull-up underwear, adult brief, baby diaper, or bed pad.

19. The substrate according to claim 16, wherein the polar organic solvent is selected from the group consisting of glycerol, propylene glycol, polypropylene glycol, polyethylene glycol, and mixtures thereof.

20. The substrate according to claim 16, wherein the cyclodextrin is an unsubstituted or substituted α-, β- or γ-cyclodextrin.

21. The substrate according to claim 16, wherein the odor-controlling compound is at least one of
an essential oil selected from the group consisting of citral, limonene, octanol, lavandin, geranium, grapefruit, linalool, citronellol, linalyl acetate, geraniol, eugenol, floracetate, cardamom, eucalyptus, juniper berry, lavender, lemon verbena, lemongrass, patchouli, rose, spearmint, tea tree, oregano, persimmon and mixtures thereof,
a botanical extract,
an odor-controlling substance selected from the group consisting of benzaldehyde, cinnamic aldehyde, ligustral, florhydral, and mixtures thereof, or
an antimicrobial substance selected from the group consisting of eucalyptus, lavender, thyme, peppermint, cajuput, cinnamon, clove, sage, tea tree oils, and mixtures thereof.

22. The substrate according to claim 16, wherein the odor-controlling solution comprises 1.5 moles to 1.75 moles of the odor-controlling compound per mole of the cyclodextrin.

23. The substrate according to claim 16, wherein the odor-controlling solution has a pH greater than or equal to 12.0.

24. An article comprising the substrate according to claim 16.

25. A method of preparing an odor-controlling substrate, comprising:
a) providing a substrate,
b) applying an odor-controlling solution onto the substrate to obtain the odor-controlling substrate,
wherein the odor-controlling solution comprises:
a polar organic solvent,
water,
a cyclodextrin, and
an odor-controlling compound, and
wherein, after b), the odor-controlling substrate comprises at least 0.2 kg of the odor-controlling compound per 1000 kg of the odor-controlling substrate.

26. The method according to claim 25, wherein applying the odor-controlling solution onto the substrate in b) increases moisture content of the substrate by less than 1 wt. %.

27. The method according to claim 25, wherein the odor-controlling solution comprises 1.5 moles to 1.75 moles of the odor-controlling compound per mole of the cyclodextrin.

28. The method according to claim 25, wherein the odor-controlling solution has a pH greater than or equal to 12.0.
